(19) United States
(12) Patent Application Publication
(10) Pub. No.: US 2009/0233979 A1
(43) Pub. Date: Sep. 17, 2009

(54) Indole Derivatives and Their Use as Thyroid Receptor Ligands

(75) Inventors: Ana Maria Garcia Collazo, Madrid (ES); Eva Kristina Koch, Stockholm (SE); Lars Johan Malm, Tumba (SE); Aiping Cheng, Huddinge (SE); Tomas Fredrik Hansson, Tumba (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: KARO BIO AB, Huddinge (SE)

(21) Appl. No.: 12/227,087

(22) PCT Filed: May 24, 2007
(86) PCT No.: PCT/EP2007/004623

§ 371 Date: Nov. 5, 2008

(30) Foreign Application Priority Data
May 24, 2006 (GB) 0610322.0

(51) Int. Cl. A61K 31/405; C07D 209/04

(52) U.S. Cl. 514/415; 548/490

(57) ABSTRACT

The invention provides compounds of formula (I) or pharmaceutically acceptable esters, amides, solvates or salts thereof, including salts of such esters or amides, and solvates of such esters, amides or salts, (I) wherein R¹, R², R³, R⁴, R⁵, R⁶, Y and W are as defined in the specification. The invention also provides the use of such compounds in the treatment or prophylaxis of a condition that may be treated with a thyroid receptor agonist or partial agonist.

### FIELD OF THE INVENTION

The present invention relates to compounds which are agonists or partial agonists of the thyroid receptor and the use of such compounds for therapeutic purposes.

### BACKGROUND OF THE INVENTION

While the extensive role of thyroid hormones in regulating metabolism in humans is well recognized, the discovery and development of new specific drugs for improving the treatment of hyperthyroidism and hypothyroidism has been slow. This has also limited the development of thyroid agonists and antagonists for treatment of other important clinical indications, such as hypercholesterolemia, dyslipidemia, obesity, diabetes, atherosclerosis, cardiac diseases, and various endocrine disorders.

Thyroid hormones affect the metabolism of virtually every cell of the body. At normal levels, these hormones maintain body weight, metabolic rate, body temperature and mood, and influence blood levels of serum lipoproteins. Thus, in hypothyroidism there is weight gain, high levels of LDL cholesterol, and depression. In hyperthyroidism, these hormones lead to weight loss, hypermetabolism, lowering of serum LDL cholesterol levels, cardiac arrhythmias, heart failure, muscle weakness, bone loss in postmenopausal women, and anxiety.

Thyroid hormones are currently used primarily as replacement therapy for patients with hypothyroidism. Therapy with thyroxine (3,5,3′,5′-tetraiodo-L-thyronine, or T₄) and triiodothyronine (3,5,3′-triiodo-L-thyronine, or T₃) returns metabolic functions to normal and can easily be monitored with routine serum measurements of levels of thyroid-stimulating hormone (TSH), T₄ or T₃. However, replacement therapy, particularly in older individuals, may be restricted by certain detrimental effects from thyroid hormones.

In addition, some effects of thyroid hormones may be therapeutically useful in non-thyroid disorders if adverse effects can be minimized or eliminated. These potentially useful influences include for example, lowering of serum LDL levels, weight reduction, amelioration of depression and stimulation of bone formation. Prior attempts to utilize thyroid hormones pharmacologically to treat these disorders have been limited by manifestations of hyperthyroidism, and in particular by cardiovascular toxicity.

Furthermore, useful thyroid agonist drugs should minimize the potential for undesired consequences due to locally induced hypothyroidism, i.e. sub-normal levels of thyroid hormone activity in certain tissues or organs. This can arise because increased circulating thyroid hormone agonist concentrations may cause the pituitary to suppress the secretion of thyroid stimulating hormone (TSH), thereby reducing thyroid hormone synthesis by the thyroid gland (negative feedback control). Since endogenous thyroid hormone levels are reduced, localized hypothyroidism can result wherever the administered thyroid agonist drug fails to compensate for the reduction in endogenous hormone levels in specific tissues.

Development of specific and selective thyroid hormone receptor ligands, particularly agonists of the thyroid hormone receptor, is expected to lead to specific therapies for these common disorders, while avoiding the cardiovascular and other toxicity of native thyroid hormones. Tissue-selective thyroid hormone agonists may be obtained by selective tissue uptake or extrusion, topical or local delivery, targeting to cells through other ligands attached to the agonist and targeting receptor subtypes. Tissue selectivity can also be achieved by selective regulation of thyroid hormone responsive genes in a tissue specific manner.

Accordingly, the compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

U.S. Pat. No. 6,794,406 discloses certain novel indole derivatives useful for treating indications which can be treated using natural thyroid hormones, for example depression, goiter or cancer of the thyroid.

We have now found that certain novel 2,3-disubstituted indole derivatives have valuable therapeutic properties.

### SUMMARY OF THE INVENTION

The present invention provides a compound of formula (I) or a pharmaceutically acceptable ester, amide, solvate or salt thereof, including a salt of such an ester or amide, and a solvate of such an ester, amide or salt,

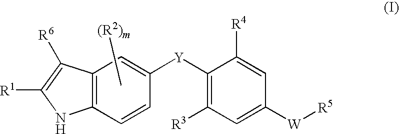

wherein:
R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—N(R^{a})₂, —(CH₂)ₙ—CO₂R^{a}, C₁₋₈ alkyl, C₂₋₄ alkenyl, C₂₋₄ alkynyl, C₃₋₆ cycloalkyl, C₃₋₆ cycloalkyl-C₁₋₃ alkyl, C₆₋₁₀ aryl, benzyl and C₃₋₇ heterocyclyl, said alkyl, alkenyl or alkynyl optionally being substituted with 1, 2 or 3 groups each independently selected from halogen, hydroxy, C₁₋₄alkylthio, phenyl, or methoxy optionally substituted with 1, 2 or 3 halogen atoms; and said cycloalkyl, C₆₋₁₀ aryl, benzyl, N(R^{a})₂ or heterocyclyl optionally being substituted with 1, 2 or 3 groups independently selected from halogen, hydroxy, cyano, C₁₋₄ alkyl, C₂₋₄ alkenyl, C₂₋₄ alkynyl, N(R^{b})₂, haloC₁₋₄alkyl, dihaloC₁₋₄alkyl, trihaloC₁₋₄alkyl or C₁₋₄alkoxy optionally substituted with 1, 2 or 3 halogen atoms, and wherein two of the 1, 2 or 3 groups may together with the atoms of the group to which they are attached form a 5-, 6- or 7-membered cyclic group optionally containing one or two heteroatoms selected from O, N and S;
each R^{a} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy;
n is 0, 1, 2 or 3;
R⁶ is selected from methyl, ethyl and n-propyl;
each R² is independently selected from halogen, mercapto, hydroxy, cyano, C₁₋₄ alkoxy, C₁₋₄ alkyl and N(R^{b})₂, said alkyl or alkoxy groups optionally being substituted with 1, 2 or 3 groups independently selected from halogen, hydroxy, methoxy, halomethoxy, dihalomethoxy, and trihalomethoxy;
each R^{b} is independently selected from a hydrogen atom and a C₁₋₄ alkyl group optionally substituted with 1, 2 or 3 groups independently selected from halogen, methoxy, halomethoxy, dihalomethoxy, and trihalomethoxy;
m is 0, 1 or 2;
Y is selected from oxygen, methylene, sulphur, SO, SO₂ and —N(R^{b})—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from C₁₋₃ alkylene, C₂₋₃ alkenylene, C₂₋₃ alkynylene, N(R^{e})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₁₋₃ alkylene and C₁₋₃ alkylene-C(O)NH—C₁₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
R^{c} is selected from hydrogen, hydroxy, C₁₋₄ alkyl, C₂₋₄ alkenyl, C₂₋₄ alkynyl, fluoromethyl, difluoromethyl and trifluoromethyl;
R⁵ is selected from —CO₂R^{d}, —PO(OR^{d})₂, —PO(OR^{d})NH₂, —SO₂OR^{d}, —COCO₂H, —CONR^{d}OR^{d}, —SO₂NHR^{d}, —NHSO₂R^{d}, —CONHSO₂R^{d}, and —SO₂NHCOR^{d};
each R^{d} is independently selected from hydrogen, C₂₋₄ alkyl, C₂A alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₋₄ alkyl.

### DETAILED DESCRIPTION OF THE INVENTION

Preferably, R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO₂R^{e}, C₁₋₈ alkyl, C₂₋₄ alkenyl, C₂₋₄ alkynyl, C₃₋₆ cycloalkyl, C₃₋₄ cycloalkyl-C₁₋₃ alkyl, phenyl, benzyl and C₃₋₇ heterocyclyl, said alkyl, alkenyl or alkynyl optionally being substituted with 1, 2 or 3 groups each independently selected from halogen, hydroxy, C₁₋₄alkylthio, phenyl, or methoxy optionally substituted with 1, 2 or 3 halogen atoms; and said cycloalkyl, phenyl, benzyl, N(R^{a})₂ or heterocyclyl optionally being substituted with 1, 2 or 3 groups independently selected from halogen, hydroxy, cyano, C₁₋₄ alkyl, C₂₋₄ alkenyl, C₂₋₄ alkynyl, N(R^{b})₂, haloC₁₋₄alkyl, dihaloC₁₋₄alkyl, trihaloC₁₋₄alkyl or methoxy optionally substituted with 1, 2 or 3 halogen atoms, and wherein two of the 1, 2 or 3 groups may together with the atoms of the group to which they are attached form a 5-, 6- or 7-membered cyclic group optionally containing one or two heteroatoms selected from O, N and S;

wherein each R^{a} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy;
R^{e} is selected from C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy; and
n is 0, 1, 2 or 3,

Any alkyl, alkenyl or alkynyl group present in R¹ is preferably unsubstituted or substituted with 1, 2 or 3 groups independently selected from halogen, hydroxy, C₁₋₄alkylthio, phenyl, methoxy, halomethoxy, dihalomethoxy, and trihalomethoxy; and any cycloalkyl, phenyl, benzyl or heterocyclyl group is preferably unsubstituted or substituted with 1, 2 or 3 groups independently selected from halogen, hydroxy, C₁₋₄ alkyl, haloC₁₋₄alkyl, dihaloC₁₋₄alkyl, tri haloC₁₋₄alkyl, methoxy, halomethoxy, dihalomethoxy, and trihalomethoxy. A substituted alkyl group R¹ may for example be a fluoromethyl, difluoromethyl or trifluoromethyl group.

Preferably, R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, —(CH₂)ₙ—CO—O—R^{a}, C₁₋₆ alkyl, C₃₋₆ cycloalkyl, C₃₋₆ cycloalkyl-C₁₋₃alkyl, phenyl, benzyl and C₃₋₇ heterocyclyl; more preferably, R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₁₋₆ alkyl, phenyl, benzyl and C₃₋₇ heterocyclyl; any alkyl, cycloalkyl, aryl or heterocyclyl group begin optionally substituted by one of the preferred substituents given above.

More preferably, R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—O—R^{a}, C₂₋₆ alkyl, C₃₋₆ cycloalkyl, C₃₋₆ cycloalkyl-C₁₋₃ alkyl, phenyl, benzyl and C₃₋₇ heterocyclyl; more preferably, R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)—NH—CO—R^{a}, C₁₋₆ alkyl, phenyl, benzyl and C₃₋₇ heterocyclyl; any alkyl, cycloalkyl, aryl or heterocyclyl group begin optionally substituted by one of the preferred substituents given above.

Preferably n is 0, 1 or 2. More preferably n is 0 or 1.

More preferably R¹ represents a C₁₋₄ alkyl group, especially a methyl group; a phenyl group optionally substituted by one or more substituents selected from halogen atoms and hydroxyl or methoxy groups; a pyridyl group; or a CONHR^{a} group.

Most preferably R¹ represents a C₂₋₄ alkyl; a phenyl group optionally substituted by one or more substituents selected from halogen atoms and hydroxy or methoxy groups; a pyridyl group; a CO₂Me group; or a CO₂Et group.

Where R¹ represents a phenyl group, the phenyl group is preferably substituted by one or two substituents selected from halogen atoms and hydroxy or methoxy groups. Preferred halogen substituents for the phenyl group are bromo, chloro or fluoro, more preferably bromo or chloro. Preferably, the phenyl group is substituted in the para or meta position with respect to the point of attachment to the indole ring, more preferably the phenyl group is substituted in the para position.

Alternatively, where R¹ represents a phenyl group, the phenyl group may be substituted with two groups which together with the atoms of the phenyl group to which they are attached form a 5-, or 6-membered cyclic group optionally containing one or two heteroatoms selected from O, N and S; more preferably the two groups together with the atoms of the phenyl group to which they are attached form a 5-, or 6-membered cyclic group, for example a 5-membered cyclic group, containing one or two oxygen heteroatoms.

Preferably R^{a} represents a C₁₋₄ alkyl, fluoromethyl, difluoromethyl, trifluoromethyl, benzyl, heterocyclyl or phenyl group being unsubstituted or substituted by C₁₋₄ alkyl or halogen. More preferably, R^{e} represents an unsubstituted C₁₋₄ alkyl, fluoromethyl, difluoromethyl, trifluoromethyl group, for example a methyl or ethyl group.

Preferably R^{e} represents a C₁₋₄ alkyl, fluoromethyl, difluoromethyl, trifluoromethyl, benzyl, heterocyclyl or phenyl, group being unsubstituted or substituted by C₁₋₄ alkyl or halogen. More preferably, R^{e} represents an unsubstituted C₁₋₄ alkyl, fluoromethyl, difluoromethyl, trifluoromethyl group, for example a methyl or ethyl group.

Preferably R⁶ represents a methyl or ethyl group, especially an ethyl group.

Preferred substituents for an alkyl or alkoxy group R² are independently selected from halogen, hydroxy, methoxy, halomethoxy, dihalomethoxy, and trihalomethoxy.

Preferably m is 1 or, especially, 0.

Preferably, if present, each R² is independently selected from halogen, hydroxy, C₁₋₄ alkoxy, C₁₋₄ alkyl and N(R^{b})₂, an alkyl or alkoxy group being unsubstituted or substituted by from 1 to 3 of the preferred substituents given above. More preferably, R² is selected from halogen, C₁₋₄ alkoxy and C₁₋₄ alkyl.

Preferably, R^{b} is selected from hydrogen and C₁₋₄ alkyl or haloalkyl.

R³ and R⁴ are preferably independently selected from halogen, C₁₋₄ alkyl, fluoromethyl, difluoromethyl and trifluoromethyl. More preferably, R³ and R⁴ are independently selected from halogen, methyl, ethyl, propyl, fluoromethyl, difluoromethyl and trifluoromethyl, especially methyl or halogen. A halogen R³ or R⁴ is preferably selected from chlorine, bromine, and fluorine, especially chlorine and bromine. Preferably R³ and R⁴ represent the same group.

Preferably, Y is methylene or oxygen, especially, oxygen.

An alkylene, alkenylene or alkynylene moiety present in W is preferably unsubstituted or substituted by 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy. Preferred halo groups are chloro or fluoro, particularly fluoro.

W is preferably selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NHC(O)—C₁₋₃ alkylene, said alkylene, alkenylene or alkynylene moiety being optionally substituted by 1 or 2 of the preferred substituents given above.

R^{e} is preferably selected from hydrogen, C₁₋₂ alkyl, fluoromethyl, difluoromethyl and trifluoromethyl.

W is more preferably selected from C₁₋₃ alkylene, C₁₋₃ alkylene-O—C₁₋₃ alkylene, C₂₋₃ alkenylene, N(R^{e})—C₁₋₂ alkylene, O—C₁₋₂ alkylene, C(O)NH—C₁₋₂ alkylene and NHC(O)—C₁₋₂ alkylene, said alkylene or alkenylene moiety optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy.

Most preferably, W is selected from C₁₋₃ alkylene, O—C₁₋₂ alkylene, C(O)NH—C₁₋₂ alkylene and NHC(O)—C₁₋₂ alkylene, especially ethylene or C(O)NH—CH₂—, in which the alkylene group (for example the ethylene group) is unsubstituted or, preferably, substituted with one or more halo groups, for example one or more fluoro groups (for example one fluoro group). Monohalo C₁₋₃ alkylene (for example fluoro C₁₋₃ alkylene) thus constitutes a preferred group W.

In another preferred embodiment, W is selected from C₁₋₃ alkylene, O—C₁₋₂ alkylene, and C(O)NH—C₁₋₂ alkylene, and monohalo C₁₋₃ alkylene. More preferably, W is selected from C₁₋₂ alkylene, O—CH₂, C(O)NH—CH₂—, and monofluoro C₁₋₂ alkylene.

In another preferred embodiment, W is selected from C₁₋₃ alkylene, C₂₋₃ alkenylene, O—C₁₋₃ alkylene, C(O)NH—C₁₋₂ alkylene and NHC(O)—C₁₋₂ alkylene.

Preferably, R⁵ is selected from —CO₂R^{d}, —SO₂OR^{d}, —COCO₂H, —CONR^{d}OR^{d}, —SO₂NHR^{d}, —NHSO₂R^{d}, —CONHSO₂R^{d}, and —SO₂NHCOR^{d};

R⁵ is more preferably selected from —CO₂R^{d}, —SO₂OR^{d}, —NHSO₂R^{d}, —COCO₂R^{d} and CONR^{d}OR^{d}. Most preferably, R⁵ is —CO₂R^{d}, or SO₂OR^{d}. Most particularly preferably, R⁵ is CO₂R^{d}, particularly —CO₂H.

R^{d} is preferably hydrogen, ethyl, methyl, phenyl or phenyl substituted with 1, 2 or 3 groups independently selected from amino, hydroxyl, halogen and methyl, particularly R^{d} is hydrogen.

Thus, in one preferred group of compounds of the invention, R¹ is selected from —(CH₂)ₙ—SO₂—R⁸, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₁₋₆ alkyl, phenyl, benzyl and C₃₋₇ heterocyclyl; any alkyl group being optionally substituted by halogen, hydroxy, phenyl, benzyl, methoxy, halomethoxy, dihalomethoxy, and trihalomethoxy, and any cycloalkyl, phenyl, benzyl or heterocyclyl group being optionally substituted with 1, 2 or 3 groups independently selected from halogen, hydroxy, C₁₋₄ alkyl, methoxy, halomethoxy, dihalomethoxy, and trihalomethoxy;

n is 0, 1 or 2, preferably 0 or 1;
R^{a} represents a C₁₋₄ alkyl, fluoromethyl, difluoromethyl, trifluoromethyl, benzyl, heterocyclyl or phenyl group being unsubstituted or substituted by C₁₋₄ alkyl or halogen.

R⁶ represents a methyl, ethyl or n-propyl group;

m is 1 or 0;
if present, R² is selected from halogen, hydroxy, C₁₋₄ alkoxy, C₁₋₄ alkyl and N(R^{b})₂, an alkyl or alkoxy group being unsubstituted or substituted by from 1 to 3 substituents independently selected from halogen, hydroxy, C₁₋₄ alkylthio, halomethoxy, dihalomethoxy, and trihalomethoxy, and R^{b} being selected from hydrogen and C₁₋₄ alkyl or haloalkyl;
R³ and R⁴ are independently selected from halogen, C₁₋₄ alkyl, fluoromethyl, difluoromethyl and trifluoromethyl;
Y is methylene or, preferably, oxygen;
W is selected from C₁₋₃ alkylene, C₁₋₃ alkylene-O—C₁₋₃ alkylene, C₂₋₃ alkenylene, N(R^{c})—C₁₋₂ alkylene, O—C₁₋₂ alkylene, C(O)NH—C₁₋₂ alkylene and NHC(O)—C₁₋₂ alkylene, said alkylene or alkenylene moieties optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy, and R^{c} being selected from hydrogen, C₁₋₂ alkyl, fluoromethyl, difluoromethyl and trifluoromethyl; and
R⁵ is selected from —CO₂H, —SO₂OR^{d}, —NHSO₂R^{d′}, —COCO₂H and CONR^{d}OR^{d} in which R^{d} is ethyl, methyl, phenyl and phenyl substituted with 1, 2 or 3 groups independently selected from amino, hydroxyl, halogen and methyl or hydrogen, particularly hydrogen.

In a further preferred group of compounds, R¹ represents a C₁₋₄ alkyl group, a phenyl group optionally substituted by one or more substituents selected from halogen atoms and hydroxy or methoxy groups, a pyridyl group, or a COOH group;

R⁶ represents a methyl, ethyl or n-propyl group, especially a methyl or ethyl group;
m is 0;
Y represents oxygen;
R³ and R⁴ represent the same group selected from halogen, methyl, ethyl, propyl, fluoromethyl, difluoromethyl and trifluoromethyl, especially methyl or halogen;
W is selected from C₁₋₃ alkylene, O—C₁₋₂ alkylene, C(O)NH—C₁₋₂ alkylene and NHC(O)—C₁₋₂ alkylene, especially ethylene or C(O)NH—CH₂—, in which the alkylene group (for example the ethylene group) is unsubstituted or, preferably, substituted with one or more halo groups, for example one or more fluoro groups (for example one fluoro group); and
R⁵ is selected from —SO₂OR^{d} in which R^{d} is ethyl, methyl or hydrogen, particularly hydrogen, or, preferably, —CO₂H.

In these preferred compounds, W is preferably monohalo C₁₋₃ alkylene (for example fluoro C₁₋₃ alkylene), C₁₋₃ alkylene, C₂₋₃ alkenylene, O—C₁₋₃ alkylene, C(O)NH—C₁₋₂ alkylene or NHC(O)—C₁₋₂ alkylene, for example CONH—CH₂, (CH₂)₂ or, especially, OCH₂ or CH₂.

The compounds of formula (I) may contain chiral (asymmetric) centres or the molecule as a whole may be chiral. The individual stereoisomers (enantiomers and diastereoisomers) and mixtures of these are within the scope of the present invention.

Suitable salts according to the invention include those formed with organic or inorganic acids or bases. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Pharmaceutically acceptable base salts include ammonium salts, alkali metal salts, for example those of potassium and sodium, alkaline earth metal salts, for example those of calcium and magnesium, and salts with organic bases, for example dicyclohexylamine and N-methyl-D-glucomine.

Pharmaceutically acceptable esters and amides of the compounds of formula (I) may have an appropriate group, for example an acid group, converted to a C₁₋₆ alkyl, C₅₋₁₀ aryl, C₅₋₁₀ aryl-C₁₋₆alkyl, or amino acid ester or amide. Pharmaceutically acceptable esters of the compounds of formula (I) may have an appropriate group, for example a hydroxy group, converted to a C₁₋₄ alkyl, C₅₋₁₀ aryl, or C₅₋₁₀aryl-C₁₋₆ alkyl ester. Pharmaceutically acceptable amides and carbamates of the compounds of formula (I) may have an appropriate group, for example an amino group, converted to a C₁₋₆ alkyl, C₅₋₁₀ aryl, C₅₋₁₀ aryl-C₁₋₆ alkyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl, and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethylbutyl, and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy. A particularly preferred alkoxy group is methoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkynyl groups include ethenyl, 1-propenyl and 2-propenyl.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo[2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₆₋₁₀ aryl is used herein to mean a group comprising from 6 to 10 carbon atoms in a monocyclic or bicyclic aromatic group. A particularly preferred C₆₋₁₀ aryl group is phenyl.

As used herein, the term “halogen” means fluorine, chlorine, bromine or iodine. Fluorine, chlorine and bromine are particularly preferred. In some embodiments, fluorine is especially preferred. In alternative embodiments, chlorine or bromine are especially preferred.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl.

As used herein the term “cycloalkylalkyl” means a group cycloalkyl-alkyl-attached through the alkyl group, “cycloalkyl” and “alkyl” being understood to have the meanings outlined above.

Numerous synthetic routes to the compounds of the present invention can be devised by any person skilled in the art and the possible synthetic routes described below are not limiting. Many methods exist in the literature for the synthesis of diaryl ethers, for example, two references directly apply to the synthesis of thyroid hormone analogs: Evans D. A. et al. Tetrahedron Lett., 39, 2937-2940, 1998 and Salamonczyk G. M. et al., Tetrahedron Lett., 38, 6965-6968, 1997.

In particular, methods for synthesizing compounds of formula I in which Y is S, and CH₂ are generally described in the literature (Y is S: Harrington, C. R., Biochem. J, 43, 434-437, 1948; Dibbo, A. et al., J. Chem. Soc., 2890-2902, 1961; Yokoyama, N. et al., U.S. Pat. No. 5,401,772, 1995; Y is CH₂: Horner, L., Medem, H. H. G., Chem. Ber., 85, 520-530, 1952; Chiellini, G. et al., Chemistry & Biology, 5, 299-306, 1998).

Compounds of the invention wherein Y contains oxygen, sulphur or —N(R^{b})— may be prepared by a process which comprises reacting a compound of formula (II)

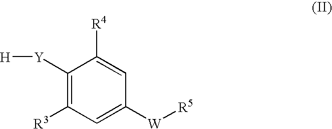

wherein W, R³, R⁴, and R⁵ are as defined above and Y is oxygen, sulphur or —N(R^{b})— with a compound of formula (III)

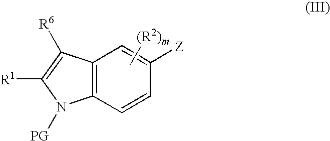

wherein R¹, R⁶, R² and m are as defined above and PG is hydrogen or a suitable protecting group, and Z is a suitable leaving group, optionally in the presence of a suitable base and optionally, in the presence of copper powder, followed optionally by removal of the protecting group, if present, and optionally by interconversion to another compound of the invention.

Suitable leaving groups Z include halogens and boron derivatives, for example a fluoride. Suitable bases include carbonates, alkylamines and alkali metal hydroxides, for example potassium carbonate, caesium carbonate, potassium hydroxide, sodium hydroxide, diisopropylamine and triethylamine. Other combinations of leaving groups and bases may be employed, as is known by the person skilled in the art. Optionally, one or more coupling reagents may be employed. The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups Y-H and Z could be switched, being the leaving group in the (II) fragment (the nucleophilic substituent, Z) and the electrophilic radical Y—H in the (III) fragment.

Preferred compounds of formula (II) include:
Methyl 3-(3,5-Dibromo-4-hydroxy-phenyl)-propanoate
Methyl (E)-3-(3,5-Dibromo-4-hydroxy-phenyl)-acrylate
Methyl (3,5-Di bromo-4-hydroxy-phenoxy)-acetate
Methyl 3-(3,5-Dibromo-4-hydroxy-phenyl)-2-fluoro-propanoate
Methyl (3,5-Dibromo-4-hydroxy-benzoylamino)-acetate

Compounds of the invention wherein Y contains methylene may be prepared by a process which comprises reacting a compound of formula (IV)

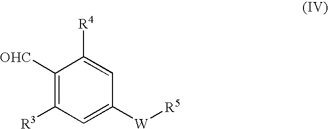

wherein W, R³, R⁴, and R⁵ are as defined above with a compound of formula (V)

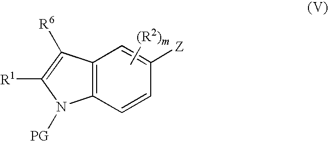

wherein R¹, R⁶, R² and m are as defined above and PG is hydrogen or a suitable protecting group, and Z may for example be lithium or a Mg-halide, such as MgBr or MgCl. Alternatively, Z may be a derivative of Sn, Pd, B or Cu.

Other combinations to produce a nucleophilic attack to an aldehyde may be employed, as is known by the person skilled in the art. Optionally, one or more coupling reagents may be employed. The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups —CHO and Z could be switched, being the leaving group in the (IV) fragment (the metal substituent, Z) and the aldehyde in the (V) fragment.

Compounds of the invention wherein Y is oxygen, sulphur or —N(R^{b})— may be prepared by reacting a compound of formula (II) in which Y is oxygen, sulphur or —N(R^{b})— with a compound of formula (VI)

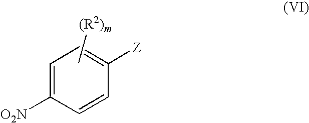

wherein R² and m are as defined above and Z is a suitable leaving group, optionally in the presence of a suitable base and optionally in the presence of copper powder, followed by reduction of the nitro group to an amino group and reaction of the resultant amine with a suitable reagent to form the bicyclic ring, followed optionally by interconversion to another compound in accordance with the invention.

Suitable leaving groups Z and various reaction conditions are as given above. Preferred compounds of formula (VI) include:
1-Fluoro-4-nitro-benzene
2-Chloro-4-fluoro-1-nitro-benzene

The invention also provides a method for preparing a compound of the invention wherein Y is oxygen, NR^{b}, sulphur or methylene comprising reacting a compound of formula (VII)

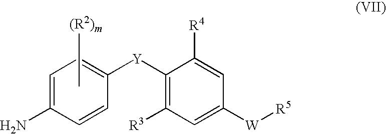

or the corresponding hydrazine of formula (VIIa) formed by reacting a compound of formula (VII) with sodium nitrate which is followed by reduction with a suitable reducing agent such as tin (II) chloride

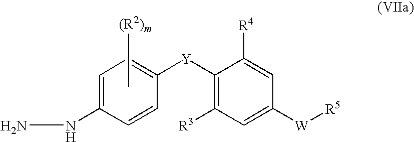

wherein R², R³, R⁴, R⁵, W and m are as defined above with a compound of formula (VIII)

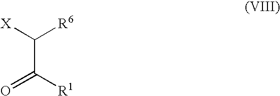

wherein R¹ and R⁶ are as defined above and X can be a halogen or a hydrogen, in the presence of a suitable acid or Lewis acid and followed optionally by interconversion to another compound of formula (I) wherein Y is oxygen, NR^{b}, sulphur or methylene.

Suitable acids for use in the reaction include acetic acid, toluenesulfonic acid and phosphorous trichloride.

Other acids may be employed, as is known by the person skilled in the art. The reaction mixture is stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999). Preferred compounds of formula (VII) include:
Methyl 3-[4-(4-amino-phenoxy)-3,5-dibromo-phenyl]-propanoate
Methyl 3-[4-(4-amino-phenoxy)-3,5-dibromo-phenyl]-2-fluoro-propanoate
Methyl (E)-3-[4-(4-amino-phenoxy)-3,5-dibromo-phenyl]-acrylate
Methyl[4-(4-amino-phenoxy)-3,5-dibromo-phenoxy]-acetate
Methyl[4-(4-amino-phenoxy)-3,5-dibromo-benzoylamino]-acetate

Preferred compounds of formula (VIII) include:
3′,4′-(methylenedioxy)butyrophenone
4-chlorobutyrophenone
p-methoxyvalerophenone
p-methoxybutyrophenone
4-propionylpyredine
2,4′-dibromopropiophenone
3-Bromo-butan-2-one
2-Bromo-1-(4-methoxy-phenyl)-propan-1-one

Compounds of the invention wherein Y is oxygen, NR^{b} or sulphur may be prepared by a process which comprises reacting a compound of formula (IX)

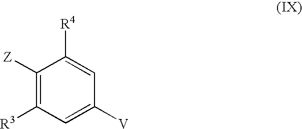

wherein R³ and R⁴ are as defined above and where substituent V represents nitro, aldehyde, cyano, carboxyl or derivatives of carboxyls and Z is a suitable leaving group, with a compound of formula (X)

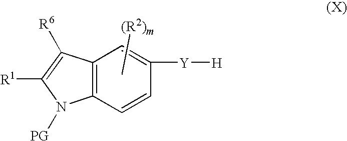

wherein R¹, R⁶, R² and m are as defined above, PG is hydrogen or a suitable protecting group, and Y is oxygen, sulphur or —N(R^{b})—, optionally in the presence of a suitable base, and optionally, in the presence of copper powder, followed optionally by removal of the protecting group, if present, and optionally by interconversion to another compound of the invention wherein Y is oxygen, NR^{b}, or sulphur.

Preferred compounds of formula (IX) include:
(3,5-Dichloro-4-fluoro-benzoylamino)-acetic acid tert-butyl ester
2-Chloro-5-nitro-1,3-bis-trifluoromethyl-benzene

Preferred compounds of formula (X) include:
5-Hydroxy-3-methyl-1H-indole-2-carboxylic acid methyl ester
5-Hydroxy-3-methyl-1H-indole-2-carboxylic acid ethyl ester
5-Hydroxy-3-methyl-1H-indole-2-carboxylic acid isopropylamide
5-Hydroxy-3-methyl-1H-indole-2-carboxylic acid ethylamide
3-Ethyl-2-(4-methoxy-phenyl)-1H-indol-5-ol
3-Ethyl-2-oxazol-4-yl-1H-indol-5-ol
2-Isobutyl-3-methyl-1H-indol-5-ol

Especially preferred compounds of formula (X) include:
5-Hydroxy-3-methyl-1H-indole-2-carboxylic acid methyl ester
5-Hydroxy-3-methyl-1H-indole-2-carboxylic acid ethyl ester
5-Hydroxy-3-methyl-1H-indole-2-carboxylic acid isopropylamide

Other suitable conditions and reagents suitable for use in the above reactions for the preparation of compounds of the invention or for the synthesis of intermediates suitable for preparing compounds of formula (I) are described in the following references:
Eur. J. Med. Chem. Chim. Ther. 31(3); 1996; 187-198
Tet. Lett. 32 (38); 1991; 5035-5038
Miyamoto, S.; J. Med. Chem.; 44, 1718-1728
B. Robinson, “The Fisher Indole synthesis”, Wiley-Interscience, NY, 1982
Organic Preparations and procedures int., 25 (6), 609-632
Gribble G. W.; Contemp. Org. Synth, 1994 p. 145-172

As mentioned above, the compounds of the invention have activity as thyroid receptor ligands. The compounds of the invention are agonists or partial agonists of the thyroid receptor. Compounds of the present invention possess activity as agonists of the thyroid receptor. They may thus be used in the treatment of diseases or disorders associated with thyroid receptor activity. In particular, compounds of the present invention may be used in the treatment of diseases or disorders associated with metabolism dysfunction or which are dependent upon the expression of a T₃ regulated gene.

The example compounds below are agonists or partial agonists of the thyroid receptor.

Clinical conditions for which an agonist or partial agonist is indicated include, but are not limited to, hypothyroidism; subclinical hyperthyroidism; non-toxic goiter; atherosclerosis; thyroid hormone replacement therapy (e.g., in the elderly); malignant tumor cells containing the thyroid receptor; papillary or follicular cancer; maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); accelerating healing of complicated fractures (e.g. distraction osteogenesis); in joint replacement; eating disorders (e.g., anorexia); treatment of obesity and growth retardation associated with obesity; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of hyperinsulinemia; stimulation of osteoblasts, bone remodeling and cartilage growth; regulation of food intake; treatment of insulin resistance, including NIDDM, in mammals (e.g., humans); treatment of insulin resistance in the heart; treatment of congestive heart failure; treatment of musculoskeletal impairment (e.g., in the elderly); improvement of the overall pulmonary function; skin disorders or diseases, such as dermal atrophy, glucocorticoid induced dermal atrophy, including restoration of dermal atrophy induced by topical glucocorticoids, and the prevention of dermal atrophy induced by topical glucocorticoids (such as the simultaneous treatment with topical glucocorticoid or a pharmacological product including both glucocorticoid and a compound of the invention), the restoration/prevention of dermal atrophy induced by systemic treatment with glucocorticoids, restoration/prevention of atrophy in the respiratory system induced by local treatment with glucocorticoids, UV-induced dermal atrophy, dermal atrophy induced by aging (wrinkles, etc.), wound healing, post surgical bruising caused by laser resurfacing, keloids, stria, cellulite, roughened skin, actinic skin damage, lichen planus, ichtyosis, acne, psoriasis, Dernier's disease, eczema, atopic dermatitis, chloracne, pityriasis and skin scarring. In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention. The term treatment includes, where appropriate, prophylactic treatment.

Accordingly, the compounds of the invention find application in the treatment or prophylaxis of the following: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

The compounds of the invention find especial application in the treatment or prophylaxis of the following: (1) hypercholesterolemia, dyslipidemia or any other lipid disorder manifested by an unbalance of blood or tissue lipid levels; (2) atherosclerosis; (3) obesity; (4) diabetes.

The invention therefore provides a method for the treatment or prophylaxis of a condition that may be treated with a thyroid receptor agonist or partial agonist in a mammal, which comprises administering to the mammal a therapeutically effective amount of a compound of the invention. Clinical conditions mediated by a thyroid receptor that may be thus treated include those described above.

The invention also provides a compound of the invention for use as a medicament, for example for use in the treatment or prophylaxis of a condition that may be treated with a thyroid receptor agonist or partial agonist. Further, the invention provides the use of a compound of the invention for the manufacture of a medicament for the treatment or prophylaxis of a condition that may be treated with a thyroid receptor agonist or partial agonist. Clinical conditions mediated by a thyroid receptor that may be treated with a thyroid receptor agonist or partial agonist include those described above.

The amount of active ingredient which is required to achieve a therapeutic effect will, of course, vary with the particular compound, the route of administration, the subject under treatment, and the particular disorder or disease being treated. The compounds of the invention may be administered orally or via injection at a dose of from 0.05 to 500 mg/kg per day, preferably 0.05 to 100 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation or composition. Accordingly, the invention provides a pharmaceutical composition comprising a compound according to the invention together with a pharmaceutically acceptable excipient.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of the invention can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug. Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an anti-dyslipidemic agent or other pharmaceutically active material.

The compounds of the present invention may be employed in combination with one or more other modulators and/or ligands of the thyroid receptor or one or more other suitable therapeutic agents selected from the group consisting of cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

Examples of suitable hypolipidemic agents for use in combination with the compounds of the present invention include an acyl coenzyme A cholesterol acyltransferase (ACAT) inhibitor, a microsomal triglyceride transfer protein (MTP) inhibitor, a cholesterol ester transfer protein (CETP) inhibitor, a ileal bile acid transporter (IBAT) inhibitor, any cholesterol absorption inhibitor, a 3-hydroxy-3-methylglutaryl coenzyme A (HMG-CoA) reductase inhibitor, a squalene synthetase inhibitor, a bile acid sequestrant, a peroxisome proliferator-activator receptor (PPAR)-alpha agonist, a peroxisome proliferator-activator receptor (PPAR)-delta agonist, any peroxisome proliferator-activator receptor (PPAR)-gamma/delta dual agonist, any peroxisome proliferator-activator receptor (PPAR)-alpha/delta dual agonist, a nicotinic acid or a derivative thereof, and a thiazolidinedione or a derivative thereof.

Examples of suitable hypolipidemic agents for use in combination with the compounds of the present invention also include ezetimibe, simvastatin, atorvastatin, rosuvastatin, cerivastatin, fluvastatin, lovastatin, pravastatin, fenofibrate, gemfibrozil and bezafibrate.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, glipyride, gliclazide, chlorpropamide and glipizide), biguanide/glyburide combinations (e.g., GlucovanceS), thiazolidinediones (e.g., troglitazone, rosiglitazone, englitazone, darglitazone and pioglitazone), PPAR-alpha agonists, PPAR-gamma agonists, PPAR alpha/gamma dual agonists, PPAR alpha/delta dual agonists, SGLT 1, 2 or 3 inhibitors, glycogen phosphorylase inhibitors, inhibitors of fatty acid binding protein (aP2), glucagon-like peptide-1 (GLP-1), glucocorticoid (GR) antagonist and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and GB98/284425 (KaroBio), CB-1 (cannabinoid receptor) antagonists (see G. Colombo et al, “Appetite Suppression and Weight Loss After the Cannabinoid Antagonist SR 141716”, Life Sciences, Vol 63, PL 113-117 (1998)) and/or an anorectic agent, such as dexamphetamine, phentermine, phenylpropanolamine or mazindol.

The compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN₇O₃ (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HTD agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CeliCept®), integrin antagonists, alpha-4 beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-KO openers such as those disclosed in U.S. Pat. No. 6,184,231 B1).

Examples of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cardiac glycosides for use in combination with the compounds of the present invention include digitalis and ouabain.

Examples of suitable cholesterol/lipid lowering agents for use in combination with the compounds of the present invention include HMG-CoA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

MTP inhibitors which may be employed herein in combination with one or more compounds of formula (I) include MTP inhibitors as disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 all incorporated herein by reference.

The HMG CoA reductase inhibitors which may be employed in combination with one or more compounds of formula (I) include mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Further HMG CoA reductase inhibitors which may be employed herein include fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0,142,146 A2, as well as other known HMG CoA reductase inhibitors.

The squalene synthetase inhibitors which may be used in combination with the compounds of the present invention include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinylmethyl)phosphonates, terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, as well as other squalene synthetase inhibitors as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

Bile acid sequestrants which may be used in combination with the compounds of the present invention include cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

ACAT inhibitors suitable for use in combination with compounds of the invention include ACAT inhibitors as described in, Drugs of the Future 24, 9-15 (1999), (Avasimibe); “The ACAT inhibitor, CI-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenyl]midazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62.

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostaglandins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

For the treatment of skin disorders or diseases as described above, the compounds of the present invention may be used alone or optionally in combination with a retinoid, such as tretinoin, or a vitamin D analog.

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Lett., 9, 1003-1008 (1999) and Hamann, L. G. et al., J. Med. Chem., 42, 210-212 (1999).

A further use of the compounds of this invention is in combination with steroidal or non-steroidal progesterone receptor agonists (“PRA”), such as levonorgestrel, medroxyprogesterone acetate (MPA).

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with a hypolipidemic agent, an antidepressant, a bone resorption inhibitor and/or an appetite suppressant, the compounds of the invention may be employed in a weight ratio to the additional agent within the range from about 500:1 to about 0.005:1, preferably from about 300:1 to about 0.01:1.

Where the antidiabetic agent is a biguanide, the compounds of the invention may be employed in a weight ratio to biguanide within the range from about 0.01:1 to about 100:1, preferably from about 0.5:1 to about 2:1.

The compounds of the invention may be employed in a weight ratio to a glucosidase inhibitor within the range from about 0.01:1 to about 100:1, preferably from about 0.5:1 to about 50:1.

The compounds of the invention may be employed in a weight ratio to a sulfonylurea in the range from about 0.01:1 to about 100:1, preferably from about 0.2:1 to about 10:1.

The compounds of the invention may be employed in a weight ratio to a thiazolidinedione in an amount within the range from about 0.01:1 to about 100:1, preferably from about 0.5:1 to about 5:1. The thiazolidinedione may be employed in amounts within the range from about 0.01 to about 2000 mg/day, which may optionally be administered in single or divided doses of one to four times per day. Further, where the sulfonylurea and thiazolidinedione are to be administered orally in an amount of less than about 150 mg, these additional agents may be incorporated into a combined single tablet with a therapeutically effective amount of the compounds of the invention.

Metformin, or salt thereof, may be employed with the compounds of formula (I) in amounts within the range from about 500 to about 2000 mg per day, which may be administered in single or divided doses one to four times daily.

The compounds of the invention may be employed in a weight ratio to a PPAR-alpha agonist, a PPAR-gamma agonist, a PPAR-alpha/gamma dual agonist, an SGLT2 inhibitor and/or an aP2 inhibitor within the range from about 0.01:1 to about 100:1, preferably from about 0.5:1 to about 5:1.

An MTP inhibitor may be administered orally with the compounds of the invention in an amount within the range of from about 0.01 mg/kg to about 100 mg/kg and preferably from about 0.1 mg/kg to about 75 mg/kg, one to four times daily. A preferred oral dosage form, such as tablets or capsules, may contain the MTP inhibitor in an amount of from about 1 to about 500 mg, preferably from about 2 to about 400 mg, and more preferably from about 5 to about 250 mg, administered on a regimen of one to four times daily. For parenteral administration, the MTP inhibitor may be employed in an amount within the range of from about 0.005 mg/kg to about 10 mg/kg and preferably from about 0.005 mg/kg to about 8 mg/kg, administered on a regimen of one to four times daily.

A HMG CoA reductase inhibitor may be administered orally with the compounds of the invention within the range of from about 1 to 2000 mg, and preferably from about 4 to about 200 mg. A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

A squalene synthetase inhibitor may be administered with the compounds of the invention within the range of from about 10 mg to about 2000 mg and preferably from about 25 mg to about 200 mg. A preferred oral dosage form, such as tablets or capsules, will contain the squalene synthetase inhibitor in an amount of from about 10 to about 500 mg, preferably from about 25 to about 200 mg.

The compounds of the invention as described above also find use, optionally in labelled form, as a diagnostic agent for the diagnosis of conditions associated with malfunction of the thyroid receptor. In particular, the compounds also find use, optionally in labelled form, as a diagnostic agent for the diagnosis of conditions that may be treated with a thyroid receptor agonist or partial agonist. For example, such a compound may be radioactively labelled.

The compounds of the invention also find use as a reference compound in methods of discovering other agonists or partial agonists of the thyroid receptor. Thus, the invention provides a method of discovering a ligand of the thyroid receptor which comprises use of a compound of the invention or a compound of the invention in labelled form, as a reference compound. For example, such a method may involve a competitive binding experiment in which binding of a compound of the invention to the thyroid receptor is reduced by the presence of a further compound which has thyroid receptor-binding characteristics, for example stronger thyroid receptor-binding characteristics than the reference compound in question.

### EXAMPLES

The following Examples illustrate the invention.

### General Experimental Conditions

Compounds were analyzed on HPLC-MS with alternating +/−API and equipped with different brands of 50 mm*2.1 mm, 5μ C8 columns. Elution was with 0.05% formic acid/acetonitrile or 0.05% ammonium acetate/acetonitrile.

MW calculated is an isotopic average and the “found mass” refers to the most abundant isotope detected in the LC-MS.

### Intermediate 1

### Methyl 3-[4-(4-aminophenoxy)-3,5-dibromophenyl]propanoate

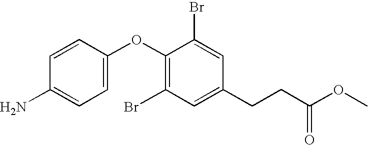

A solution of p-fluoro nitrobenzene (210 mg, 1.5 mmol), methyl 3-(4-hydroxy-3,5-dibromophenyl)propanoate (500 mg, 1.5 mmol) and potassium carbonate (410 mg, 3 mmol) in dimethylsulfoxide (3 mL) was purged with nitrogen and heated at 130° C. for 17 h. The mixture was diluted with ethylacetate and washed with sodium bicarbonate (sat), water and brine. The combined organic phases were evaporated on silica and purified by flash chromatography (heptane/ethyl acetate 10:0 to 5:5) to give methyl 3-[3,5-d]bromo-4-(4-nitrophenoxy)phenyl]propanoate as a white solid (504 mg, yield: 74%).

To a stirred solution of methyl 3-[3,5-d]bromo-4-(4-nitrophenoxy)phenyl]propanoate (505 mg, 1.1 mmol) in acetic acid (25 mL) and water (3 mL), iron powder (308 mg, 5.5 mmol) was added. The reaction mixture was stirred for 17 h at 20° C. Acetic acid was removed under vacuum and the residue was diluted with ethyl acetate (50 mL) and water (50 mL) and extracted with ethyl acetate (2×5 mL). The combined ethyl acetate layers were washed with brine, dried over sodium sulphate and concentrated. The residue was purified by flash chromatography (dichloromethane/methanol 10:0 to 9:1) to afford the title compound (310 mg) in 72% yield (MW=429.1). LC/MS (ESI): m/z 430.4 (M+1).

### Intermediate 2

### [4-(4-Amino-phenoxy)-3,5-dichloro-phenyl]-acetic acid methyl ester

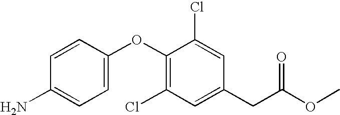

A stirred solution of 2,6-dichlorophenol (8.1 g, 50 mmol) in acetonitrile (40 mL) was cooled to 0° C. and bromine (9.6 g) in acetonitrile (10 mL) was added dropwise. The red solution was stirred at 0° C. for 2 h and a saturated aqueous solution of sodium sulphite was added until the red colour disappeared. The phases were separated and the aqueous phase was extracted three times with ethyl acetate. Concentration of the combined organic phases gave a yellow oil, which was purified on a silica gel column (heptane/ethyl acetate, 10:1) to give 11.33 g of 4-Bromo-2,6-dichloro-phenol as a white solid. (Yield: 95.5%)

A stirred solution of 4-Bromo-2,6-dichloro-phenol (5.76 g, 20 mmol), 4-fluoronitrobenzene (2.82 g, 20 mmol), potassium carbonate (5.5 g, 48 mmol) and copper powder (128 mg, 2 mmol) in DMF (40 mL) was heated at 135° C. for 45 h. The reaction was cooled to room temperature and concentrated. The residue was dissolved in ethyl acetate and washed twice with sodium hydroxide (2 M), twice with hydrochloric acid (1 M) and brine. After concentration of the organic phase, the residue was purified on a silica gel column (heptane: 100%) to give 5 g of 5-Bromo-1,3-dichloro-2-(4-nitro-phenoxy)-benzene as white solid. (Yield: 69%).

To a solution of 5-Bromo-1,3-dichloro-2-(4-nitro-phenoxy)-benzene (1.8 g, 5 mmol), Pd(Ph₃P)Cl₂ (80 mg, 0.1 mmol) and CuI (40 mg, 0.2 mmol) was added triethylamine (1 g, 10 mmol), followed by trimethylsilylacetylene (735 mg, 7.5 mmol). The reaction mixture was stirred under N₂ at 60° C. for 1 h. The reaction mixture was cooled to room temperature, filtrated and concentrated. The residue was dissolved in ethyl acetate and the organic phase was washed twice with water and once with brine. After concentration of the organic phase, the residue was purified on column (heptane/ethyl acetate, 10:1) to give 1.8 g (94.9%) of [3,5-dichloro-4-(4-nitro-phenoxy)-phenylethynyl]-trimethyl-silane as a white solid.

Cyclohexene (3.4 g) was added dropwise to a solution of borane (1 M in THF, 21 mL, 21 mmol) at 0° C. [3,5-Dichloro-4-(4-nitro-phenoxy)-phenylethynyl]-trimethyl-silane (2.4 g, 6 mmol) in THF was added dropwise at 0° C. and the reaction mixture was stirred at this temperature for 2 h. A mixture of sodium hydroxide (17 ml, 1 M) and methanol (20 mL) was added dropwise at 0° C. followed by dropwise addition of hydrogen peroxide (10 mL, 33%, 97 mmol) at the same temperature. The mixture was stirred at 0° C. for an additional hour and concentrated. The remaining aqueous solution was acidified with hydrochloric acid (IM) and extracted three times with ethyl acetate. Concentration of the organic phase gave a dark oil which was used in the next step without further purification. (4.87 g, crude)

The crude product above was dissolved in methanol (100 ml) and thionyl chloride (0.1 mL) was carefully added. The mixture was stirred at reflux for two hours. The reaction mixture was concentrated; water was added and extracted three times with ethyl acetate. Purification on column (silica, heptane/ethyl acetate, 10:1) gave 1.2 g of [3,5-Dichloro-4-(4-nitro phenoxy)-phenyl]-acetic acid methyl ester as white solid. (Yield: 56% of two steps)

To a solution of [3,5-dichloro-4-(4-nitro phenoxy)-phenyl]-acetic acid methyl ester in ethyl acetate was added platinum oxide monohydrate and the mixture was stirred vigorously under H₂ atmospheres for 2 h. The suspension was filtered and the filtrate concentrated. The residue was purified on column (silica gel, petroleum ether/ethyl acetate, 4:1) to give 0.96 g of [4-(4-Amino-phenoxy)-3,5-dichloro-phenyl]-acetic acid methyl ester as white solid. (Yield: 89.5%).

### Intermediate 3

### Methyl 3-[4-(4-aminophenoxy)-3,5-dichlorophenyl]propanoate

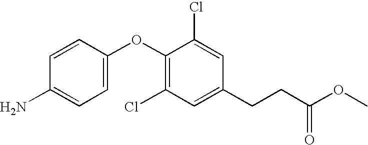

A solution of p-fluoro nitrobenzene (500 mg, 3.5 mmol), methyl 3-(4-hydroxy-3,5-dichlorophenyl)propanoate (500 mg, 2 mmol), copper (243 mg, 3.8 mmol) and potassium carbonate (630 mg, 4.5 mmol) in dimethylformamide (7 mL) was heated at 100° C. for 3 h. The cooled reaction mixture was diluted with ethyl acetate and hydrochloric acid (1M) and extracted with ethyl acetate (3×10 mL). The combined organic phases were concentrated and filtered through a short flash chromatography column (heptane/ethyl acetate 10:0 to 5:5) to give methyl 3-[3,5-dichloro-4-(4-nitrophenoxy)phenyl]propanoate (600 mg, yield: 80%).

To a stirred solution of methyl 3-[3,5-dichloro-4-(4-nitrophenoxy)phenyl]propanoate (600 mg, 1.6 mmol) in ethanol (50 mL) and water (3 mL), tin dichloride (1.83 g, 8 mmol) was added. The reaction mixture was stirred for 17 h at 90° C. Ethanol was removed under vacuum and the residue was diluted with ethyl acetate (50 mL) and saturated solution of sodium carbonate (50 mL) and extracted with ethyl acetate (2×10 mL). The combined ethyl acetate layers were washed with brine, dried over sodium sulphate and concentrated. The residue was purified by flash chromatography (heptane/ethyl acetate 10:0 to 7:3) to afford the title compound (180 mg) in 35% yield.

### Intermediate 4

### Methyl 2-fluoro-3-[3,5-dibromo-4-(4-aminophenoxy)phenyl]propanoate

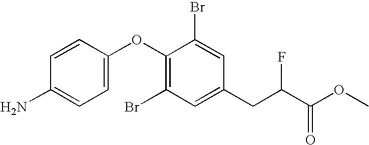

Sodium hydride (242 mg, 7.06 mmol, 70%) was dissolved in dry methanol (30 mL) with stirring. Methyl 2-hydroxy-3-(4-hydroxy-3,5-dibromophenyl) propanoate (2.5 g, 7.06 mmol) was added to the solution at room temperature and the solvent was evaporated off under reduced pressure to give the sodium phenolate as a white solid.

The phenolate and p-dinitrobenzene (1.19 g, 7.06 mmol) were dissolved in dimethyl sulphoxide (25 mL). The reaction mixture was heated to 90° C. for 15 h under a calcium chloride guard tube. The reaction mixture was poured into ice-water (150 mL) and extracted with ethyl acetate (3×100 mL). The combined organic phases were washed with sodium hydroxide (50 mL, aqueous 1 M) and brine and dried over sodium sulphate, filtrated and evaporated. The crude was purified by chromatography on silica gel (ethyl acetate/petroleum 1:9 to 2:8) to afford 1.39 g of methyl 2-hydroxy-3-[3,5-dibromo-4-(4-nitrophenoxy)phenyl]propanoate (43% yield).

A solution of methyl 2-hydroxy-3-[3,5-d]bromo-4-(4-nitrophenoxy)phenyl]propanoate (1.39 g, 2.93 mmol) in dry dichloromethane (15 mL) was added dropwise to the solution of DAST (Et₂NSF₃) (0.5 g, 3.13 mmol) in dry dichloromethane (10 mL) at 0° C. under nitrogen atmosphere. The mixture was stirred for 15 min and allowed to come to room temperature and poured into a mixture of ice and water. The organic layer was separated and the water was extracted with dichloromethane (2×60 mL). The combined organic layers were washed with brine and dried over sodium sulphate, filtrated and evaporated. Methyl 2-fluoro-3-[3,5-d]bromo-4-(4-nitrophenoxy)phenyl]propanoate (1.62 g) was pure enough to be used in the next step without further purification. Pd/C (10%, 80 mg) was added to a solution of methyl 2-fluoro-3-[3,5-d]bromo-4-(4-nitrophenoxy)phenyl) propanoate (1.62 g) in methanol (150 mL). The reaction mixture was stirred under H₂ at room temperature until the starting material disappeared. The mixture was filtered through celite and the solvent evaporated under vacuum. The residue was purified by chromatography on silica gel (ethyl acetate/heptane 2:8 to 3:7) to afford 1.15 g of methyl 2-fluoro-3-[3,5-d]bromo-4-(4-aminophenoxy)phenyl]propanoate as a yellow solid (80% yield for two steps).

### Intermediate 5

### (3,5-Dichloro-4-fluoro-benzoylamino)-acetic acid tert-butyl ester

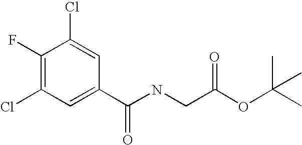

3,5-Dichloro-4-fluoro-benzoic acid (1,036 g, 5 mmol), N,N-1-(3-dimethylaminopropyl)-3-ethyl-carbodiimide hydrochloridethyl (1.319 g, 5 mmol), 1-hydroxybenzotriazole hydrate (1,048 mg, 8.5 mmol), and 1971 μL of triethylamine were stirred at 0° C. in DMF(35 ml). Glycine tert-butyl ester hydrochloride (1.676 g, 10 mmol) was added and the reaction mixture was stirred at 0° C. for 0.5 h. The reaction was allowed to reach room temperature and was stirred at room temperature for 65 h. The reaction was quenched with 5 ml of water and the solvents were removed by evaporation. To the remaining water phase, 200 ml of ethyl acetate was added. The organic layer was washed with brine (3×50 mL) and dried over Na₂SO₄. After evaporation, the residue was dissolved in 50 mL acetone, and 12 mL of ethyl acetate and 38 mL of heptane were added. After careful evaporation to remove acetone and cooling, the organic phase was filtered to remove formed precipitate. The precipitate was washed with 50 ml of mixture solvent (ethyl acetate/heptane 25:75). The combined organic phases were evaporated and the residue was purified with flash chromatography (ethyl acetate/heptane 40:60) to give 1.165 g of (3,5-dichloro-4-fluoro-benzoylamino)-acetic acid tert-butyl ester.

### Intermediate 6

### Methyl 14-(4-aminophenoxy)-3,5-dibromophenoxy]acetate

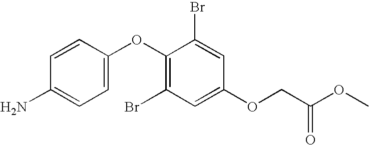

Sodium methoxide (2.2 g, 40 mmol) was added to a solution of 1,3-dibromo-5-fluoro-2-(4-nitrophenoxy)benzene (4 g, 10 mmol) in dimethylformamide (15 mL) at room temperature. The mixture was stirred at room temperature for 4 h. Water (20 mL) was added to the mixture and the product was extracted with ethyl acetate. The combined organic phases were washed consecutively with diluted hydrochloric acid and brine, dried over anhydrous magnesium sulphate and concentrated in vacuo. This crude mixture was used immediately without further purification. Boron trifluoride-methyl sulfide complex (1M, 12.8 mL, 12.8 mmol) was added dropwise to a stirred, chilled (dry ice-acetone bath) solution of crude 1,3-dibromo-5-methoxy-2-(4-nitrophenoxy)benzene (4.9 g, 12 mmol) in dichloromethane (150 mL). The mixture was allowed to warm up to room temperature and was stirred overnight. The reaction mixture was concentrated under vacuum, diluted with water, and extracted with ethyl acetate. The combined organic phases were washed with diluted hydrochloric acid, saturated sodium bicarbonate and brine, dried over anhydrous magnesium sulphate and concentrated in vacuo. The residue was purified by flash chromatography (petroleum ether/ethyl acetate 20:1) to give 2.5 g (64.3%) of 3,5-dibromo-4-(4-nitrophenoxy)phenol as light yellow oil.

Ethyl bromoacetate (2.5 mL, 22 mmol) was added to a mixture of 3,5-dibromo-4-(4-nitrophenoxy)phenol (5.2 g, 13 mmol) and potassium carbonate (7.6 g, 54 mmol) in acetone (150 mL) at 0° C. After being stirred at ambient temperature for 4 h, the mixture was concentrated in vacuo. Ethyl acetate was added to the residue and the organic phase was washed with brine, dried over anhydrous magnesium sulphate and concentrated in vacuo to give the crude mixture of ethyl[3,5-d]bromo-4-(4-nitrophenoxy)phenoxy]acetate which was used without further purification. To a solution of ethyl[3,5-d]bromo-4-(4-nitrophenoxy)phenoxy]acetate (3.8 g, 8 mmol) in ethanol (150 mL), tin(II) chloride (9 g, 47 mmol) was added and the reaction mixture was stirred overnight at 80° C. After cooling to room temperature, the mixture was concentrated in vacuo and ethyl acetate and water were added to the residue. The organic phase was washed with sodium hydroxide (25% aqueous) and brine, dried over anhydrous potassium carbonate and concentrated in vacuo. The residue was purified by flash chromatography (petroleum ether/ethyl acetate 4:1 to 2:1) to give 1.2 g (64.3%) of methyl[4-(4-aminophenoxy)-3,5-dibromophenoxy]acetate.

### Intermediate 7

### 4-(4-Amino-phenoxy)-3,5-dibromo-benzoic acid methyl ester

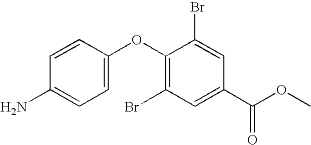

Bromine (5.75 ml, 2.05 eq) in glacial acetic acid (80 mL) was added dropwise into a solution of p-cresol (5.9 g, 54.6 mmol.) in acetic acid (12 mL) and water (33 mL) with stirring and temperature control with a water-bath. The reaction solution was stirred for additional 0.5 h at room temperature and poured into water (200 mL). The precipitate was collected and purified by recrystallisation from ethyl acetate/heptane and 13.2 g of 2,6-Dibromo-4-methyl-phenol was obtained (yield 91%). Sodium hydride (0.46 g, 13.4 mmol) was pre-washed with hexane and was carefully dissolved in methanol (anhydrous, 41 mL 2,6-Dibromo-4-methyl-phenol (3.43 g, 12 mmol) was added to the basic solution, and the mixture was evaporated to obtain the sodium salt as a white solid. Anhydrous DMSO (18.5 mL) and p-dinitrobenzene (1.90 g, 11.3 mmol) was mixed with the sodium salt, and heated at 90° C. for 16 h. The reaction solution was poured into 500 mL of water/ice, and extracted with diethyl ether (3×500 mL). The combined organic phases were washed with 5% aqueous sodium hydroxide (200 mL), water (200 mL), dried, filtrated and concentrated. The residue was purified on silica chromatograph to give 2.6 g of 1,3-dibromo-5-methyl-2-(4-nitro-phenoxy)-benzene. (Yield: 59%).

1,3-Dibromo-5-methyl-2-(4-nitro-phenoxy)-benzene (2.60 g, 6.7 mmol) was dissolved in pyridine (30 mL) and water (12 mL), and heated to reflux. KMnO₄ (8.5 g, 53.8 mmol) was added in portions while the mixture was refluxed, and heated for additional 6 h. The reaction mixture was cooled to ambient temperature and the solution was diluted with ethyl acetate and filtered with celite. Concentration of the solution gave a sticky solid which was diluted with 2M HCl and extracted with ethyl acetate. The organic phase was concentrated and dissolved in 5% NaOH, which was washed with ether. The alkaline solution was acidified with conc. HCl and extracted with acetate. The combined organic phases were dried and concentrated to give 2.0 g of 3,5-Dibromo-4-(4-nitro-phenoxy)-benzoic acid. (Yield: 71%).

3,5-Dibromo-4-(4-nitro-phenoxy)-benzoic acid (1.1 g) was refluxed in methanol (100 mL) with a catalytic amount of toluenesulfonic acid for 2 days to give 1.0 g of 3,5-dibromo-4-(4-nitro-phenoxy)-benzoic acid methyl ester. (Yield: 85%).

3,5-Dibromo-4-(4-nitro-phenoxy)-benzoic acid methyl ester (900 mg, 2.1 mmol) and PtO₂ (60 mg, 0.1 eq.) were suspended in ethyl acetate (60 mL), and the reaction mixture was put under H₂ atmospheres for 16 h. The reaction solution was filtered, concentrated and recrystallized from acetate/petroleum to obtain 630 mg of 4-(4-amino-phenoxy)-3,5-dibromo-benzoic acid methyl ester (yield: 75%)

### General Procedure A for the Preparation of Examples 1-5, 19 and 20

To a solution of the appropriate aniline (e.g. methyl 3-[4-(4-aminophenoxy)-3,5-dichlorophenyl]acetate, intermediate 2) (2 eq.) in n-butanol (20 ml/mmol) was added the appropriate ketone (e.g. β-bromo-2-butanone) (1 eq.) and acetic acid (1.2 eq.). The mixture was heated to 130° C. under positive pressure of nitrogen for 17 h. The solution was diluted with ethyl acetate, extracted with ethyl acetate and the combined organic phases washed with water and evaporated. The residue was purified by flash chromatography (dichloromethane/ethyl acetate 5:5). The obtained ester intermediate was dissolved in a 1:1 mixture of tetrahydrofuran and lithium hydroxide (1 M) (20 mL/mmol) and stirred for 17 h at room temperature. The reaction mixture was acidified to pH 2 with hydrochloric acid (2M) and extracted with ethyl acetate. The combined organic phases were evaporated and purified by semi-preparative HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) to yield the wanted product (e.g. {3,5-dibromo-4-[(2,3-dimethyl-1H-indol-5-yl)oxy]phenyl}acetic acid).

### General Procedure B for the Preparation of Examples 6-13 and 15

To a stirred solution of an appropriate aniline (e.g. methyl N-[4-(4-aminophenoxy)-3,5-dibromobenzoate) in concentrated hydrochloric acid (20 mL/mmol) and acetic acid (4 mL/mmol) at 0° C., was added sodium nitrite (1.1 eq.). After 1 h stirring at 0° C., tin(II) chloride (2.5 eq.) in concentrated hydrochloric acid (4 mL/mmol) was added dropwise. After 1 h at 0° C., the yellow precipitate formed was filtered and washed with hydrochloric acid (2M) and water. The filtrate was dissolved in acetic acid (32 mL/mmol) and hydrochloric acid (4 μL/mmol) and the appropriate ketone (e.g. 4-chlorobutyrophenone) (1.1 eq.) was added. The mixture was heated at 40° C. for 1 h, followed by 17 h at 70° C. After evaporation of the acetic acid, the crude was dissolved in a 1:1 mixture of tetrahydrofuran and lithium hydroxide (1 M) (20 mL/mmol) and stirred for 17 h. The mixture was acidified to pH 3 with hydrochloric acid (2M) and extracted with ethyl acetate. The combined organic phases were evaporated and purified by semi-preparative HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) to give the wanted product (e.g. 3,5-dibromo-4-{[2-(4-methoxyphenyl)-3-methyl-1H-indol-5-yl]oxy}benzoic acid).

### Example 14

### N-(3,5-dibromo-4-{[2-(4-chlorophenyl)-3-ethyl-1H-indol-5-yl]oxy)benzoyl)glycine

3,5-Dibromo-4-{[2-(4-chlorophenyl)-3-ethyl-1H-indol-5-yl]oxy}benzoic acid (6 mg, 0.01 mmol) which was made utilising general procedure B from intermediate 7, glycine methyl ester (hydrochloride salt, 5 mg, 0.04 mmol), 3-ethyl-]-[3-(dimethylamino)propyl]carbodiimide hydrochloride (EDCI) (5 mg, 0.03 mmol) and 1-hydroxybenzotriazole hydrate (HOBt) (4 mg, 0.03 mmol) were dissolved in anhydrous dichloromethane (0.7 mL). After the addition of triethylamine (4 μL, 0.03 mmol), the reaction mixture was stirred overnight at room temperature. The solution was diluted with ethyl acetate, extracted (3×5 mL), washed with water and evaporated. The crude was filtered through a silica SPE column and eluted with dichloromethane. The obtained ester intermediate was dissolved in a 1:1 mixture of tetrahydrofuran and lithium hydroxide (1 M) (20 mL/mmol) and stirred for 17 h. The reaction mixture was acidified to pH 3 with hydrochloric acid (2M) and extracted with ethyl acetate (3×10 mL). The combined organic phases were evaporated and purified by semi-preparative HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) giving N-(3,5-dibromo-4-{[2-(4-chlorophenyl)-3-ethyl-1H-indol-5-yl]oxy}benzoyl)glycine as a slightly yellow solid (yield 18%)

### Example 16

To a stirred solution of the 5-Methoxy-3-methyl-1H-indole-2-carboxylic acid (42 mg, 0.125 mmol), made by basic hydrolysis from the commercially available 5-Methoxy-3-methyl-1H-indole-2-carboxylic acid ethyl ester (purchased from Zerenex Molecular Ltd, UK), in DMF (3 ml) was successively added 1-hydroxybenzotriazole hydrate (60 mg, 0390 mmol) and N,N-1-(3-dimethylaminopropyl)-3-ethyl-carbodiimide hydrochloridethyl (75 mg, 0.390 mmol) and isopropylamine (40 mg, 0.39 mmol) at 0° C. After being stirred for 1 h at 0° C. the mixture was allowed to reach ambient temperature and was left stirring for 17 h. After filtration of the mixture, the filtrate was evaporated in vacuo. Purification on a silica SPE column (heptane:EtOAc 7:3) gave 5-methoxy-3-methyl-1H-indole-2-carboxylic acid isopropylamide as a solid. The indole was dissolved in dichloromethane (10 mL) which was cooled to 0° C. and put under nitrogen atmosphere. Boron trifluoride dimethyl sulphide complex (3 equivalents) was added. The organic phase was washed with water, brine, dried over Na₂SO₄, filtrated and evaporated. The crude was utilised without further purification. The crude (30 mg) containing 5-Hydroxy-3-methyl-1H-indole-2-carboxylic acid isopropylamide, 1,3-Dichloro-2-fluoro-5-nitro-benzene (22 mg, 0.1 mmol) and K₂CO₃ (23 mg, 0.17 mmol) was dissolved in DMSO (1.5 mL), purged with N₂ and stirred at 120° C. for 17 h. The mixture was diluted with EtOAc and washed with a saturated Na₂CO₃ solution, water, and brine. The organic phase were evaporated on silica and purified on a silica column (heptane: EtOAc, 7:3) to give 20 mg of 5-(2,6-dichloro-4-nitro-phenoxy)-3-methyl-1H-indole-2-carboxylic acid isopropylamide as a solid.

The biaryl indole was dissolved in a mixture of BOAc and H₂O (9:1, 5 mL) and Fe (27 mg) was added and the reaction was left stirring at ambient temperature for 3 h. The reaction was diluted with EtOAc and HCl (2M) was added. The organic phase was separated and the water phase extracted twice with EtOAc. The combined organic phases were washed with brine, dried with Na₂SO₄, filtrated and evaporated. The evaporation gave 17 mg as a brown oil which was used without further purification.

To the crude (17 mg) containing 5-(4-amino-2,6-dichloro-phenoxy)-3-methyl-1H-indole-2-carboxylic acid isopropylamide, which was dissolved in CH₂Cl₂ (10 mL) and cooled to 0° C., was added ethyl malonyl chloride (11 μL, 0.087 mmol) and pyridine (9 μL, 0.11 mmol). After 2.5 h at 0° C. the reaction was quenched by addition of HCl (3 mL, 1 M). After stirring for 10 min, additional CH₂Cl₂ (30 mL) and HCl (1 mL, 1 mL) was added. The organic layer was washed with HCl (1M), H₂O, brine and evaporated. The crude was dissolved in THF (5 ml) and LiOH (1M, 2 ml) and left overnight. The reaction mixture was acidified to pH 3 with HCl (2M). EtOAc was added and the organic phase was washed with brine, dried with Na₂SO₄, filtrated and evaporated. The ethyl ester was dissolved in THF (5 ml) and LiOH (1M, 2 ml) was added. The reaction was left stirring for 4 days at ambient temperature. HCl (2M) was added until pH 3 was reached. Extraction with EtOAc (2×20 ml), washed with brine, dried with Na₂SO₄ and evaporated. The crude was purified by semi-preparative HPLC (Zorbax CombiHT (SB-C8 50×21.2 mm, 51) Mobile Phase: Solvent A. Water with 0.5% formic acid; Solvent B: acetonitrile. Gradient: 2 min 80% of A then over 8 min to 5% of A) giving 2.5 mg as a solid (yield: 4.1%) LCMS.

### General Procedure C for the Preparation of Examples 17 and 18

A solution of 5-Hydroxy-3-methyl-1H-indole-2-carboxylic acid methyl ester (21 mg, 0.1 mmol), (3,5-Dichloro-4-fluoro-benzoylamino)-acetic acid tert-butyl ester (Intermediate 5, 33 mg, 0.1 mmol) and K₂CO₃ (28 mg, 0.2 mmol) in DMF (2 mL) was purged with nitrogen and heated to 120° C. for 17 h.

Ethyl acetate (30 mL) was added and the organic layer was washed 3 times with brine, dried over Na₂SO₄ and evaporated. The residue was purified on a silica column (ethyl acetate/heptene 2:3), to give 31 mg of 5-[4-(tert-butoxycarbonylmethyl-carbamoyl)-2,6-dichloro-phenoxy]-3-methyl-1H-indole-2-carboxylic acid methyl ester. 5-[4-(tert-Butoxycarbonylmethyl-carbamoyl)-2,6-dichloro-phenoxy]-3-methyl-1H-indole-2-carboxylic acid methyl ester (31 mg, mmol) was dissolved in a mixture of acetic acid: iso-propanol:H₂O (4:4:1, 2 mL), and stirred at 100° C. for 15 hr.

After evaporation of the solvent, the residue was purified by semi-preparative HPLC (Zorbax CombiHT (SB-C8 50×21.2 mm, 51) Mobile Phase: Solvent A. Water with 0.5% formic acid; Solvent B: acetonitrile. Gradient: 2 min 80% of A then over 8 min to 5% of A) giving 13 mg of 5-[4-(carboxymethyl-carbamoyl)-2,6-dichloro-phenoxy]-3-methyl-1H-indole-2-carboxylic acid methyl ester (yield 28.8%).

### Example 21

2-Chloro-5-nitro-1,3-bis-trifluoromethyl-benzene (purchased from ABCR GmbH & Co, 107 mg, 0.36 mmol) and K₂CO₃ (101 mg, 0.73 mmol) were added to a solution of 5-hydroxy-3-methyl-1H-indole-2-carboxylic acid ethyl ester (80 mg, 0.36 mmol) in acetone (4 mL) under a positive pressure of nitrogen. The mixture was heated to 55° C. and stirred for 4 hr until the starting materials had been consumed. Ethyl acetate (50 ml) was added and the organic layer was separated, washed with brine (3×30 mL), dried over Na₂SO₄, filtrated and evaporated. The residue was purified on a silica column (ethyl acetate/heptene 40:60) giving 162 mg of 3-methyl-5-(4-nitro-2,6-bis-trifluoromethyl-phenoxy)-1H-indole-2-carboxylic acid ethyl ester. (Yield: 93.2%).

To a solution of 3-methyl-5-(4-nitro-2,6-bis-trifluoromethyl-phenoxy)-1H-indole-2-carboxylic acid ethyl ester (162 mg, 0.34 mmol) in ethanol (50 mL), platinum oxide (7 mg, cat) was added. The mixture was evacuated and put under a positive pressure of hydrogen (4 psi) at room temperature for 6 hr. The reaction mixture was filtered via celite to remove catalyst, and evaporated to give 145 mg of crude product containing 5-(4-amino-2,6-bis-trifluoromethyl-phenoxy)-3-methyl-1H-indole-2-carboxylic acid ethyl ester which was utilised without further purification. 70 mg of the crude mixture containing 5-(4-amino-2,6-bis-trifluoromethyl-phenoxy)-3-methyl-1H-indole-2-carboxylic acid ethyl ester was dissolved with CH₂Cl₂ (10 mL) and methyl malonyl chloride (19 μl, 0.16 mmol) was added. The mixture was cooled to 0° C. for 10 minutes and triethylamine (22 μl, 0.16 mmol) in CH₂Cl₂ (2 mL) was added with a syringe. After the addition the reaction mixture was stirred at 0° C. for 3 h. The reaction was quenched by addition of a saturated aqueous solution of ammonium chloride (2 mL). The organic phases were separated and evaporation gave 82 mg containing 5-[4-(2-methoxycarbonyl-acetylamino)-2,6-bis-trifluoromethyl-phenoxy]-3-methyl-1H-indole-2-carboxylic acid ethyl ester, which was dissolved in 1,4-dioxane (2 mL), and NaOH (1 M, 3 mL) was added. The mixture stirred at room temperature for 17 h.

The pH was adjusted to 4-5 with HCl (IM) and the mixture was extracted with ethyl acetate (3×30 mL). The organic layer was washed with brine (2×20 mL), water (2×20 mL) and evaporated. Purification with semi-preparative HPLC (Zorbax CombiHT (SB-C8 50×21.2 mm, 5μ) Mobile

Phase: Solvent A. Water with 0.5% formic acid; Solvent B: acetonitrile. Gradient: 2 min 80% of A then over 8 min to 5% of A) giving 51 mg of 5-[4-(2-Carboxy-acetylamino)-2,6-bis-trifluoromethyl-phenoxy]-3-methyl-1H-indole-2-carboxylic acid in an overall yield of 67.8%.

The full chemical names of the compounds of Examples 1 to 21 are as follows, and the structures are summarized in the following Table:
(3,5-dichloro-4-{[2,3-dimethyl-1H-indol-5-yl]oxy}phenyl)acetic acid
(3,5-dichloro-4-{[2-(4-methoxyphenyl)-3-methyl-1H-indol-5-yl]oxy}phenyl)acetic acid
3-(3,5-dibromo-4-{[2-(4-methoxyphenyl)-3-methyl-1H-indol-5-yl]oxy}phenyl)propanoic acid
(3,5-dibromo-4-{[2-(4-methoxyphenyl)-3-methyl-1H-indol-5-yl]oxy}phenoxy)acetic acid
3-{3,5-dibromo-4-[(2,3-dimethyl-1H-indol-5-yl)oxy]phenyl}propanoic acid
(4-{[2-(4-bromophenyl)-3-methyl-1H-indol-5-yl]oxy}-3,5-dichlorophenyl)acetic acid
(3,5-dichloro-4-{[2-(4-hydroxyphenyl)-3-methyl-1H-indol-5-yl]oxy}phenyl)acetic acid
3-(4-{[2-(1,3-benzodioxol-5-yl)-3-ethyl-1H-indol-5-yl]oxy}-3,5-dichlorophenyl)propanoic acid
3-(4-{[2-(1,3-benzodioxol-5-yl)-3-ethyl-1H-indol-5-yl]oxy}-3,5-dibromophenyl)propanoic acid
3-{3,5-Dibromo-4-[(3-methyl-2-pyridin-4-yl-1H-indol-5-yl)oxy]phenyl}propanoic acid
3-(3,5-dibromo-4-{[2-(4-methoxyphenyl)-3-ethyl-1H-indol-5-yl]oxy}phenyl)propanoic acid
3-(3,5-dibromo-4-{[2-(4-chlorophenyl)-3-ethyl-1H-indol-5-yl]oxy}phenyl)propanoic acid
3-(3,5-dibromo-4-{[2-(4-methoxyphenyl)-3-propyl-1H-indol-5-yl]oxy}phenyl)propanoic acid
N-(3,5-dibromo-4-{[2-(4-chlorophenyl)-3-ethyl-1H-indol-5-yl]oxy}benzoyl)glycine

3-{3,5-Dibromo-4-[3-ethyl-2-(4-methoxy-phenyl)-1H-indol-5-yloxy]-phenyl}-2-fluoro-propionic acid
N-[3,5-Dichloro-4-(2-isopropylcarbamoyl-3-methyl-1H-indol-5-yloxy)-phenyl]-malonamic acid
5-[4-(Carboxymethyl-carbamoyl)-2,6-dichloro-phenoxy]-3-methyl-1H-indole-2-carboxylic acid methyl ester
5-[4-(Carboxymethyl-carbamoyl)-2,6-dichloro-phenoxy]-3-methyl-1H-indole-2-carboxylic acid ethyl ester
3-[3,5-Dichloro-4-(2,3-dimethyl-1H-indol-5-yloxy)-phenyl]-propionic acid
3-{3,5-Dichloro-4-[2-(4-methoxy-phenyl)-3-methyl-1H-indol-5-yloxy]-phenyl}-propionic acid
5-[4-(2-Carboxy-acetylamino)-2,6-bis-trifluoromethyl-phenoxy]-3-methyl-1H-indole-2-carboxylic acid

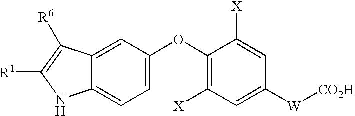

| | | | | | Yield | MW | M + 1 |
|---|---|---|---|---|---|---|---|
| Example | R¹ | R⁶ | X | W | (%) | (calc) | (found) |
| 1 | Me | Me | Cl | CH₂ | 11 | 364.2 | 364.4 |
| 2 | p-OMe-Ph | Me | Cl | CH₂ | 6 | 456.3 | 456.5 |
| 3 | p-OMe-Ph | Me | Br | (CH₂)₂ | 20 | 559.2 | 560.3 |
| 4 | p-OMe-Ph | Me | Br | O—CH₂ | 4 | 561.2 | 562.0 |
| 5 | Me | Me | Br | (CH₂)₂ | 11 | 467.2 | 468.2 |
| 6 | p-Br-Ph | Me | Cl | CH₂ | 34 | 505.2 | 506.0 |
| 7 | p-HO-Ph | Me | Cl | CH₂ | 11 | 442.3 | 443.2 |
| 8 | p, m-OCH₂O-Ph | Et | Cl | (CH₂)₂ | 1 | 498.4 | 498.5 |
| | | | | | | | M |
| 9 | p, m-OCH₂O-Ph | Et | Br | (CH₂)₂ | 3 | 587.3 | 588.2 |
| 10 | p-OMe-Ph | Et | Br | (CH₂)₂ | 8 | 573.3 | 574.4 |
| 11 | p-Cl-Ph | Et | Br | (CH₂)₂ | 11 | 577.3 | 578.6 |
| 12 | Py | Me | Br | (CH₂)₂ | 9 | 530.2 | 531.2 |
| 13 | p-OMe-Ph | n-Pr | Br | (CH₂)₂ | 1 | 587.3 | 588.2 |
| 14 | p-Cl-Ph | Et | Br | CONH—CH₂ | 18 | 606.7 | 607.1 |
| 15 | p-OMe-Ph | Et | Br | CH₂—CHF | 7.6 | 591.3 | 592.4 |
| 16 | CONHCH(CH₃)₂ | Me | Cl | NHCO—CH₂ | 4.1 | 478.3 | 478.1 |
| 17 | —CO₂Me | Me | Cl | CONH—CH₂ | 28.8 | 451.3 | 451.2 |
| 18 | —CO₂Et | Me | Cl | CONH—CH₂ | 23.5 | 465.3 | 465.2 |
| 19 | Me | Me | Cl | (CH₂)₂ | 46.3 | 378.3 | 378.5 |
| 20 | p-OMe-Ph | Me | Cl | (CH₂)₂ | 47.8 | 470.4 | 470.6 |
| 21 | —CO₂H | Me | CF₃ | NHCO—CH₂ | 67.8 | 504.3 | 505.8 |

The following compounds are further Examples of the invention:

| Example | Structure | Name |
|---|---|---|
| 22 | 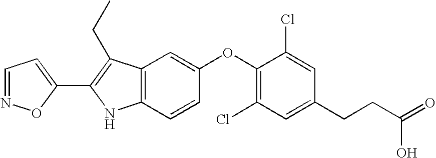 | 3-[3,5-Dichloro-4-(3-ethyl-2- isoxazol-5-yl-1H-indol-5- yloxy)-phenyl]-propionic acid |
| 23 | 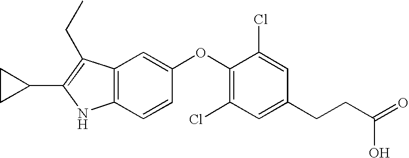 | 3-[3,5-Dichloro-4-(2- cyclopropyl-3-ethyl-1H-indol- 5-yloxy)-phenyl]-propionic acid |
| 24 | 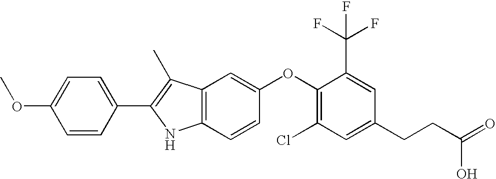 | 3-{3-Chloro-4-[2-(4-methoxy- phenyl)-3-methyl-1H-indol-5- yloxy]-5-trifluoromethyl- phenyl}-propionic acid |
| 25 | 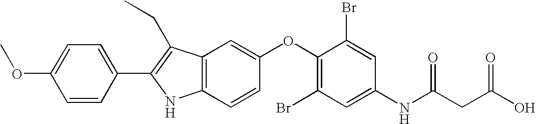 | N-{3,5-Dibromo-4-[3-ethyl-2- (4-methoxy-phenyl)-1H-indol- 5-yloxy]-phenyl}-malonamic acid |
| 26 | 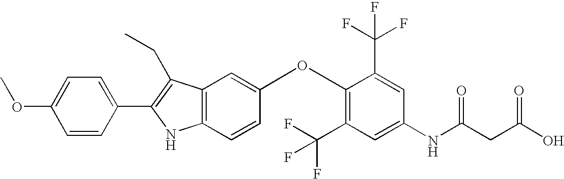 | N-{4-[3-Ethyl-2-(4-methoxy- phenyl)-1H-indol-5-yloxy]-3,5- bis-trifluoromethyl-phenyl}- malonamic acid |
| 27 | 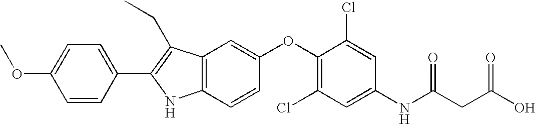 | N-{3,5-Dichloro-4-[3-ethyl-2- (4-methoxy-phenyl)-1H-indol- 5-yloxy]-phenyl}-malonamic acid |
| 28 | 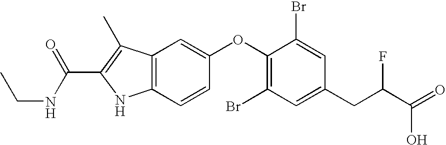 | 3-[3,5-Dibromo-4-(2- ethylcarbamoyl-3-methyl-1H- indol-5-yloxy)-phenyl]-2- fluoro-propionic acid |
| 29 | 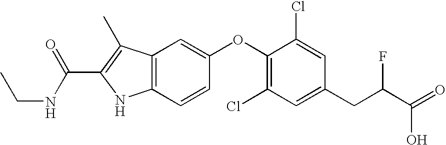 | 3-[3,5-Dichloro-4-(2- ethylcarbamoyl-3-methyl-1H- indol-5-yloxy)-phenyl]-2- fluoro-propionic acid |
| 30 | 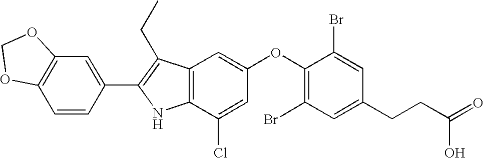 | 3-[3-Chloro-4-(2- ethylcarbamoyl-3-methyl-1H- indol-5-yloxy)-5- trifluoromethyl-phenyl]-2- fluoro-propionic acid |
| 31 | 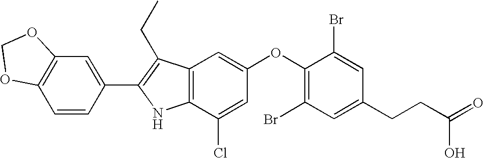 | 3-[4-(2-Benzo[1,3]dioxol-5-yl- 7-chloro-3-ethyl-1H-indol-5- yloxy)-3,5-dibromo-phenyl]- propionic acid |
| 32 | 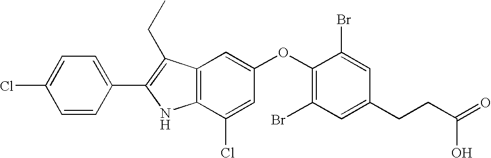 | 3-{3,5-Dibromo-4-[7-chloro-2- (4-chloro-phenyl)-3-ethyl-1H- indol-5-yloxy]-phenyl}- propionic acid |

### Procedures for the Preparation of Comparative Examples

### Comparative Example 1

To a solution of the aniline (Intermediate 3) in ethanol (5 ml), which completely dissolved after sonification, was added HCl (conc., 2.5 ml). The reaction mixture was cooled in an ice-bath and vigorously stirred. When the HCl salt was formed (precipitate) the reaction mixture was put in an EtOH/CO₂-ice bath and a solution of sodium nitrate (21 mg) in H₂O (1 ml) was added slowly and the reaction mixture was kept at −70° C. for 10 min. After the addition the flask was put in an ice bath for 0.5 h. A cooled solution of stannous chloride (198 mg) in HCl (1.5 ml) was added slowly. After 4 h at 0° C. a white precipitate was formed. Shaken between diethyl ether (3×50 ml) and a potassium hydroxide solution (25%).

The combined organic phases were dried over Na₂SO₄ and evaporated. The brown oil was dissolved in MeOH (10 ml), the 3-methyl-butyraldehyde and a catalytic amount of H₂SO₄ was added. The flask was heated to 80° C. for 17 h. EtOAc and H₂O was added and the organic phase was washed with brine. The organic phase was dried over Na₂SO₄, filtration and evaporation, gave a brown oil which was purified on a silica column (Heptane:EtOAc, 9:1-8:2). The collected fractions were evaporated and gave 40 mg as a slightly yellow oil.

10 mg of the indole ester was dissolved in THF (1 ml). LiOH (1 ml, 1 M) was added. The reaction mixture was left at room temperature with stirring for 17 h. The reaction was acidified with HCl (2M) to pH˜2 and extracted with CHCl₃. Separation with a phase separator and purification by preparative HPLC. Evaporation of the collected fractions gave 4 mg of 3-[3,5-dichloro-4-(3-isopropyl-1H-indol-5-ylmethyl)-phenyl]-propionic acid a white solid.

### Comparative Example 2

3-[3,5-Dichloro-4-(3-isopropyl-1H-indol-5-ylmethyl)-phenyl]-propionic acid methyl ester (indole ester intermediate isolated prior to the final step in the synthesis of Comparative Example 1, 20 mg, 0.05 mmol) was dissolved in 3 mL of CH₂Cl₂. NBS (9 mg, 0.05 mmol) was added and the reaction is left stirring under N₂-atmosphere at room temperature for 4 h. The crude was evaporated and put on a SPE silica column (Heptane EtOAc 9:1). Evaporation of the collected fractions gave 14 mg as brown oil that solidified. A solution containing 3-[4-(2-bromo-3-isopropyl-1H-indol-5-yloxy)-3,5-dichloro-phenyl]-propionic acid methyl ester (14 mg, 0.03 mmol) in DME (1 mL) was prepared and treated with aqueous K₂CO₃ (13 mg in 0.5 mL H₂O) and phenyl boronic acid (14 mg, 0.09 mmol). The mixture was stirred at 60° C. for 6 h. The mixture was allowed to reach room temperature and then filtered through celite. The filtrate was diluted with EtOAc, washed with brine and dried. Concentration in vacuo afforded a residue which was dissolved in THF (2 mL) and LiOH (1 M, 1 mL) and left stirring at room temperature for 17 h. After acidification to pH 3, the mixture was extracted with EtOAc, which was evaporated. Purification with semi-preparative HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) giving 1.2 mg of 3-{3,5-Dichloro-4-[3-isopropyl-2-(4-methoxy-phenyl)-1H-indol-5-yloxy]-phenyl}-propionic acid as a white solid in an overall yield of 4.8%.

Comparative examples 3 and 4 correspond to examples 3 and 5 respectively of U.S. Pat. No. 6,794,406 and may be synthesised according to the methods described in U.S. Pat. No. 6,794,406.

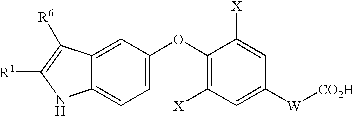

| Compara- | | | | | | | |
|---|---|---|---|---|---|---|---|
| tive | | | | | Yield | MW | M + 1 |
| Example | R¹ | R⁶ | X | W | (%) | (calc) | (found) |
| 1 | H | Iso- | Cl | (CH₂)₂ | 17.3 | 392.3 | 392.2 |
| | | Pr | | | | | |
| 2 | p-OMe-Ph | Iso- | Cl | (CH₂)₂ | 4.8 | 498.4 | 498.8 |
| | | Pr | | | | | |
| 3 | H | Iso- | Me | NH—CH₂ | 9.5 | 380.5 | 381.5 |
| | | Pr | | (Ethyl ester | | | |
| | | | | of acid) | | | |
| 4 | H | Iso- | Me | NH—CH₂ | 5.1 | 352.4 | 353.8 |
| | | Pr | | | | | |

TR Competition Binding Assay with Filter Separation

Compounds are tested for their ability to compete with the tracer ¹²⁵I-T3 for binding to full length hTRα and hTRβ. Receptor extracts and tracer are diluted in assay buffer (17 mM K₂HPO₄, 3 mM KH₂PO₄, 400 mM KCl, 1 mM MgCl₂, 0.5 mM EDTA and 8.7% glycerol). ¹²⁵I-T3 is diluted to a final concentration of 0.2 nM and receptor is diluted to reach a final count in Trilux Microbeta of approximately 10000 ccpm. Compounds are typically serially diluted in DMSO from DMSO stock solutions of 10 mM. To 96 well microtiter plates are 100 μl tracer, 4 μl test compound dilution series and 100 μl receptor dilution added. The assay plates are incubated at +4° C. over night (app. 16 hrs incubation). Receptor bound and free tracer are separated over a glass fiber filter (FILTERMAT B, PerkinElmer)) on a Tomtec Cellharvester with 18 mM K₂HPO₄, 2 mM KH2PO₄, 0.5 mM EDTA wash buffer. The filters are dried at 60° C. for 1 hour and then merged with a scintillant wax (MELTILEX, PerkinElmer) on a Wallac Microsealer before measuring in a Trilux Microbeta. IC50s, the concentration test compound needed to decrease tracer binding by 50 percent, are generated via analysis of data in XLfit version 2.0 or later with a four parameter logistic model. Compounds are considered to have activity as thyroid receptor-beta ligands if they have an IC₅₀ of 500 nM or less. Preferred thyroid receptor-beta ligand compounds have an IC₅₀ of less than 100 nM, especially less than 30 nM. Particularly preferred as thyroid receptor-beta ligands are those compounds having an IC₅₀ of 10 nM or less.

### Vector Constructs, Generation of Reporter Cell Lines (TRAF), and Assay Procedure.

The cDNAs encoding the full length human ThRα1 and ThRβ1 were cloned in the mammalian expression vector pMT-hGH. The pDR4-ALP reporter vector contains one copy of the direct repeat sequence AGGTCA nnnnAGGTCA, fused upstream of the core promoter sequences of the mouse mammary tumor virus long terminal repeat (MMTV), replacing the glucocorticoid response elements. The DR4-MMTV promoter fragment was then cloned in the 5′ end of the cDNA encoding human placental alkaline phosphatase (ALP), followed in the 3′-end by the polyA-signal sequence of the human growth hormone gene. Chinese hamster ovary (CHO) K1 cells (ATCC No. CCL 61) were transfected in two steps, first with the receptor expression vectors pMT-hThRα1 and pMT-ThRβ1, respectively, and the drug resistance vector pSV2-Neo, and in the second step, with the reporter vector pDR4-ALP and the drug resistance vector pKSV-Hyg. Individual drug resistant clones were isolated and selected based on T3 inducibility. One stable reporter cell clone each of CHO/hThRα1 and CHO/hThRβ1 were chosen for further study in response to various thyroid hormone agonists.

### Assay Procedure:

CHO/hThRα1 and CHO/hThRβ1 were seeded in growth medium (Coon's/F12, 10% L-3,5,3′-triiodothyronine and L-thyroxine depleted FCS, 2 mM L-glutamine) in 96-well plates at 20×10³ cells per well. After 24 hour incubation at 37° C. in humidified chambers, at 5% CO₂, conditioned medium was replaced by induction medium (OptiMEM, 2 mM L-glutamine, 50 μm/ml gentamycin) and cells were exposed to the test compounds at serial dilutions, at final DMSO concentration of 0.5%, or to serial dilution of T3 (positive control), to assess agonist activity of test compounds.

In order to examine antagonistic effect of test compounds, CHO/hThRα1 and CHO/hThRα1 cells were exposed to serial dilution of the compounds in the presence of 1 nM T3 (CHO/hThRα1) or 3 nM T3 (CHO/hThRβ1).

After 48 hours incubation at 37° C. in humidified chambers at 5% CO₂ the level of alkaline phosphatase expressed and secreted into the cell culture medium was analyzed by chemiluminescence on MicroBeta Trilux.

Compounds are considered to be agonists of the thyroid receptor-beta ligands if they exhibit agonism of at least 50% and display no antagonism.

The assay procedures described above were carried out for the compounds of Examples 1-21 and Comparative Examples 1-4. Selected results for exemplary compounds are given in table below.

| | TR-β IC50 | |
|---|---|---|
| Compound | (nM) | β % agonism |
| 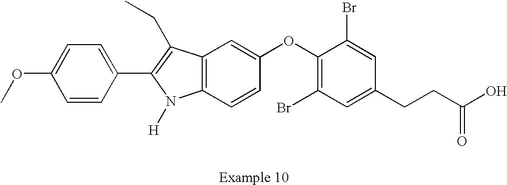 | 2.77 | 79.73 |
| 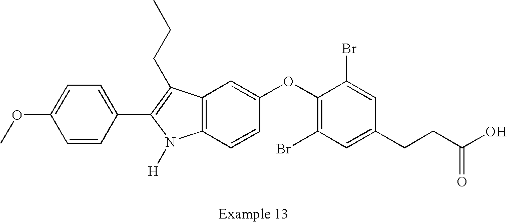 | 8.88 | 70.65 |
| 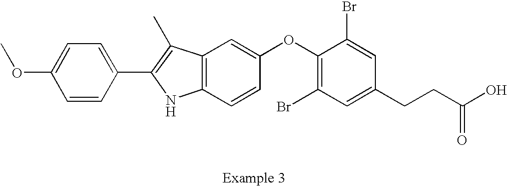 | 7.16 | 97.50 |
| 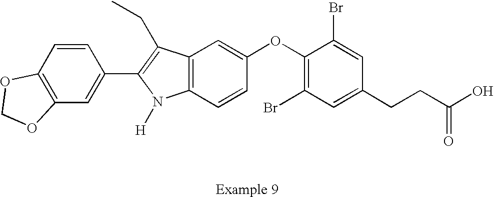 | 0.33 | 70.35 |
| 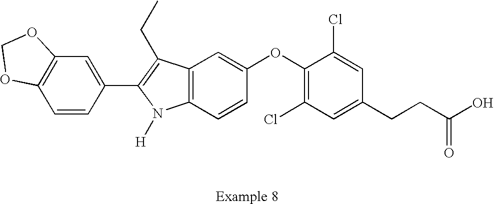 | 2.5 | 76.93 |
| 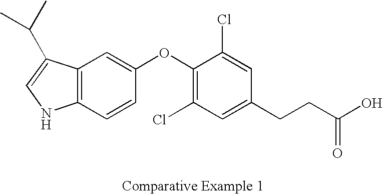 | 19.15 | 66.20 |
| 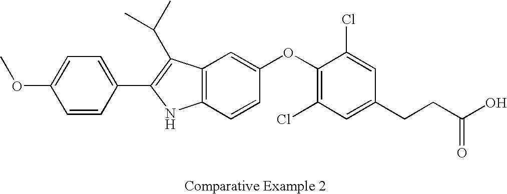 | 51.54 | 80.20 |
| 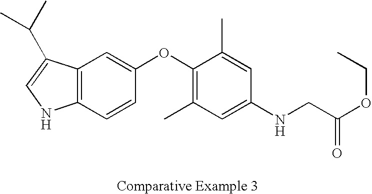 | 326.18 | 69.70 |
| 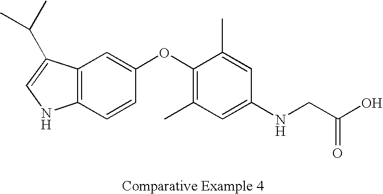 | 43.06 | 39.70 |

As shown in the Table above, introducing a substituent into the 2-position on the indole ring (Examples 10, 13, 3, 9, 8) leads to improved binding to the TR-β receptor and/or improved agonistic activity when compared to compounds lacking a substituent in the 2-position on the indole ring (Comparative Examples 1, 3, 4).

It is hypothesized that this improvement is due to the unexpected flexibility of the binding cavity in the thyroid hormone receptor ligand binding domain allowing the accommodation of ligands with surprisingly large R¹ substituents.

1. A compound of formula (I) or a pharmaceutically acceptable ester, amide, solvate or salt thereof, including a salt of such an ester or amide, and a solvate of such an ester, amide or salt, 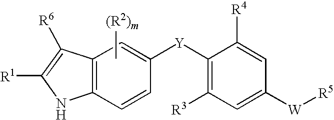
wherein:
R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂) N—CO—N(R^{a})₂, —(CH₂) n-CO₂R^{a}, C₁₋₈ alkyl, C₂₋₄ alkenyl, C₂₋₄ alkynyl, C₃₋₆ cycloalkyl, C₃₋₆ cycloalkyl-C₁₋₃ alkyl, C₆₋₁₀ aryl, benzyl and C₃₋₇ heterocyclyl, said alkyl, alkenyl or alkynyl optionally being substituted with 1, 2 or 3 groups each independently selected from halogen, hydroxy, C₁₋₄alkylthio, phenyl, or methoxy optionally substituted with 1, 2 or 3 halogen atoms; and said cycloalkyl, C₆₋₁₀ aryl, benzyl, N(R^{a})₂ or heterocyclyl optionally being substituted with 1, 2 or 3 groups independently selected from halogen, hydroxy, cyano, C₁₋₄ alkyl, C₂₋₄ alkenyl, C₂₋₄ alkynyl, N(R^{b})₂, haloC₁₋₄alkyl, dihaloC₁₋₄alkyl, trihaloC₁₋₄alkyl or C₁₋₄alkoxy optionally substituted with 1, 2 or 3 halogen atoms, and wherein two of the 1, 2 or 3 groups may together with the atoms of the group to which they are attached form a 5-, 6- or 7-membered cyclic group optionally containing one or two heteroatoms selected from O, N and S;
each R^{a} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy;
n is 0, 1, 2 or 3;
R⁶ is selected from methyl, ethyl and n-propyl;
each R² is independently selected from halogen, mercapto, hydroxy, cyano, C₁₋₄ alkoxy, C₁₋₄ alkyl and N(R^{b})₂, said alkyl or alkoxy groups optionally being substituted with 1, 2 or 3 groups independently selected from halogen, hydroxy, methoxy, halomethoxy, dihalomethoxy, and trihalomethoxy;
each R^{b} is independently selected from a hydrogen atom and a C₁₋₄ alkyl group optionally substituted with 1, 2 or 3 groups independently selected from halogen, methoxy, halomethoxy, dihalomethoxy, and trihalomethoxy;
m is 0, 1 or 2;
Y is selected from oxygen, methylene, sulphur, SO, SO₂ and —N(R^{b})—;
R³ and R⁴ are independently selected from halogen, C₁₋₄ alkyl, fluoromethyl, difluoromethyl, trifluoromethyl, C₁₋₄ alkoxy, fluoromethoxy, difluoromethoxy and trifluoromethoxy;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NHC(O)—C₁₋₃ alkylene and C₁₋₃ alkylene-C(O)NH—C₁₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
R^{c} is selected from hydrogen, hydroxy, C₁₋₄ alkyl, C₂₋₄ alkenyl, C₂₋₄ alkynyl, fluoromethyl, difluoromethyl and trifluoromethyl;
R⁵ is selected from —CO₂R^{d}, —PO(OR^{d})₂, —PO(OR^{d})NH₂, —SO₂OR^{d}, —COCO₂H, —CONR^{d}OR^{d}, —SO₂NHR^{d}, —NHSO₂R^{d}, —CONHSO₂R^{d}, and —SO₂NHCOR^{d};
each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₋₄ alkyl.

2. A compound as claimed in claim 1, wherein R¹ is selected from —(CH₂)ₙ—SO₂—R^{a}, —(CH₂)ₙ—NH—SO₂—R^{a}, —(CH₂), —SO₂—NH—R^{a}, —(CH₂)ₙ—NH—CO—R^{a}, —(CH₂)ₙ—CO₂R^{e}, C₁₋₈ alkyl, C₂₋₄ alkenyl, C₂₋₄ alkynyl, C₃₋₆ cycloalkyl, C₃₋₆ cycloalkyl-C₁₋₃ alkyl, phenyl, benzyl and C₃₋₇ heterocyclyl, said alkyl, alkenyl or alkynyl optionally being substituted with 1, 2 or 3 groups each independently selected from halogen, hydroxy, C₁₋₄alkylthio, phenyl, or methoxy optionally substituted with 1, 2 or 3 halogen atoms; and said cycloalkyl, phenyl, benzyl, N(R^{a})₂ or heterocyclyl optionally being substituted with 1, 2 or 3 groups independently selected from halogen, hydroxy, cyano, C₁₋₄ alkyl, C₂₋₄ alkenyl, C₂₋₄ alkynyl, N(R^{b})₂, haloC₁₋₄alkyl, dihaloC₁₋₄alkyl, trihaloC₁₋₄alkyl or methoxy optionally substituted with 1, 2 or 3 halogen atoms, and wherein two of the 1, 2 or 3 groups may together with the atoms of the group to which they are attached form a 5-, 6- or 7-membered cyclic group optionally containing one or two heteroatoms selected from O, N and S;
each R^{a} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy;
R^{e} is selected from C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy, and trihalomethoxy; and
n is 0, 1, 2 or 3.

3. A compound as claimed in claim 2, wherein R¹ is selected from a phenyl group optionally substituted by one, two or three substituents selected from halogen atoms and hydroxy or methoxy groups, and wherein two of the 1, 2 or 3 substituents may together with the atoms of the phenyl group to which they are attached form a 5-, 6- or 7-membered cyclic group optionally containing one or two heteroatoms selected from O, N and S; a pyridyl group; a CO₂Me group; or a CO₂Et group.

4. A compound as claimed in claim 1, wherein R⁶ is methyl or ethyl.

5. A compound as claimed in claim 1, wherein R³ and R⁴ are independently selected from halogen, C₁₋₄ alkyl, fluoromethyl, difluoromethyl and trifluoromethyl.

6. A compound as claimed in claim 1, wherein W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NHC(O)—C₁₋₃ alkylene, said alkylene, alkenylene or alkynylene moiety being optionally substituted by 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy.

7. A compound as claimed in claim 6, wherein W is selected from C₁₋₃ alkylene, O—C₁₋₂ alkylene, and C(O)NH—C₁₋₂ alkylene, and monohalo C₁₋₃ alkylene.

8. A compound as claimed in claim 1, wherein R⁵ is selected from —CO₂R^{d}, —SO₂OR^{d}, —NHSO₂R^{d′}, —COCO₂R^{d} and CONR^{d}OR^{d}.

9. A compound as claimed in claim 1, wherein R¹ is selected from —(CH₂)ₙ—SO₂—R^{a}, —(CH₂)ₙ—NH—SO₂—R^{a}, —(CH₂)ₙ—SO₂—NH—R^{a}, —(CH₂)ₙ—NH—CO—R^{a}, —(CH₂)ₙ—CO—NH—R^{a}, C₁₋₆ alkyl, phenyl, benzyl and C₃₋₇ heterocyclyl; any alkyl group being optionally substituted by halogen, hydroxy, phenyl, benzyl, methoxy, halomethoxy, dihalomethoxy, and trihalomethoxy, and any cycloalkyl, phenyl, benzyl or heterocyclyl group being optionally substituted with 1, 2 or 3 groups independently selected from halogen, hydroxy, C₁₋₄ alkyl, methoxy, halomethoxy, dihalomethoxy, and trihalomethoxy;
n is 0, 1 or 2;
R^{a} represents a C₁₋₄ alkyl, fluoromethyl, difluoromethyl, trifluoromethyl, benzyl, heterocyclyl or phenyl group being unsubstituted or substituted by C₁₋₄ alkyl or halogen.
R⁶ represents a methyl, ethyl or n-propyl group;
m is 1 or 0;
if present, R² is selected from halogen, hydroxy, C₁₋₄ alkoxy, C₁₋₄ alkyl and N(R^{b})₂, an alkyl or alkoxy group being unsubstituted or substituted by from 1 to 3 substituents independently selected from halogen, hydroxy, C₁₋₄ alkylthio, halomethoxy, dihalomethoxy, and trihalomethoxy, and R^{b} being selected from hydrogen and C₁₋₄ alkyl or haloalkyl;
R³ and R⁴ are independently selected from halogen, C₁₋₄ alkyl, fluoromethyl, difluoromethyl and trifluoromethyl;
Y is methylene or oxygen;
W is selected from C₁₋₃ alkylene, C₁₋₃ alkylene-O—C₁₋₃ alkylene, C₂₋₃ alkenylene, N(R^{c})—C₁₋₂ alkylene, O—C₁₋₂ alkylene, C(O)NH—C₁₋₂ alkylene and NHC(O)—C₁₋₂ alkylene, said alkylene or alkenylene moieties optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy, and R^{c} being selected from hydrogen, C₁₋₂ alkyl, fluoromethyl, difluoromethyl and trifluoromethyl; and
R⁵ is selected from —CO₂H, —SO₂OR^{d}, —NHSO₂R^{d}, —COCO₂H and CONR^{d}OR^{d} in which R^{d} is ethyl, methyl, phenyl and phenyl substituted with 1, 2 or 3 groups independently selected from amino, hydroxyl, halogen and methyl or hydrogen, particularly hydrogen.

10. (canceled)

11. (canceled)

12. A method for the treatment or prophylaxis of a condition that may be treated with a thyroid receptor agonist or partial agonist in a mammal, which comprises administering to the mammal a therapeutically effective amount of a compound of formula (I) as defined in claim 1 or a pharmaceutically acceptable ester, amide, solvate or salt thereof, including a salt of such an ester or amide, and a solvate of such an ester, amide or salt.

13. (canceled)

14. A pharmaceutical composition comprising a compound of formula (I) as defined in claim 1 or a pharmaceutically acceptable ester, amide, solvate or salt thereof, including a salt of such an ester or amide, and including a solvate of such an ester, amide or salt, and a pharmaceutically acceptable excipient.

15. A pharmaceutical composition as claimed in claim 14 further comprising an additional therapeutic agent selected from cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

16. Use of a compound of formula (I) as defined in claim 1 in labelled form as a diagnostic agent for the diagnosis of conditions that may be treated with a thyroid receptor agonist or partial agonist.

17. Use of a compound of formula (I) as defined in claim 1 or a labelled form of such a compound as a reference compound in a method of identifying ligands for the thyroid hormone receptor.

18. A method as claimed in claim 12, wherein the condition that may be treated with a thyroid receptor agonist or partial agonist is selected from (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

19. A process for preparing a compound of formula (I) as defined in claim 1 wherein Y is oxygen, sulphur or —N(R^{b})—, comprising reacting a compound of formula (II) 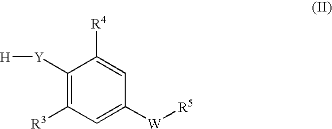
wherein W, R³, R⁴, and R⁵ are as defined in claim 1 and Y is oxygen, sulphur or —N(R^{b})—, with a compound of formula (III) 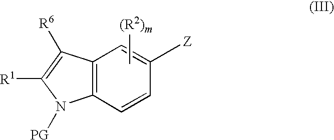
wherein R¹, R⁶, R² and m are as defined in claim 1 and PG is hydrogen or a suitable protecting group, and Z is a suitable leaving group, optionally in the presence of a suitable base and optionally, in the presence of copper powder, followed optionally by removal of the protecting group, if present, and optionally by interconversion to another compound of formula (I) as defined in claim 1 wherein Y is oxygen, sulphur or —N(R^{b})—.

20. A process for preparing a compound of formula (I) as defined in claim 1 wherein Y is methylene, comprising reacting a compound of formula (IV) 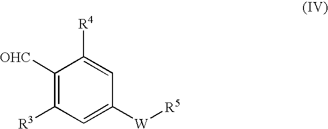
wherein W, R³, R⁴, and R⁵ are as defined in claim 1 with a compound of formula (V) 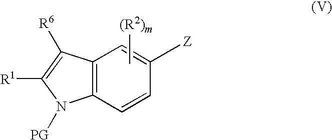
wherein R¹, R⁶, R² and m are as defined in claim 1 and PG is hydrogen or a suitable protecting group, and Z is lithium, MgBr, MgCl, or a derivative of Sn, Pd, B or Cu, followed optionally by removal of the protecting group, if present, and optionally by interconversion to another compound of formula (I) as defined in claim 1 wherein Y is methylene.

21. A process for preparing a compound of formula (I) as defined in claim 1 wherein Y is oxygen, sulphur or —N(R^{b})—, comprising reacting a compound of formula (II) as defined in claim 19 in which Y is oxygen, sulphur or —N(R^{b})—, with a compound of formula (VI) 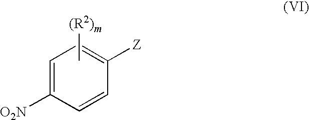
wherein R² and m are as defined in claim 1 and Z is a suitable leaving group, optionally in the presence of a suitable base and optionally in the presence of copper powder, followed by reduction of the nitro group to an amino group and reaction of the resultant amine with a suitable reagent to form the bicyclic ring, followed optionally by interconversion to another compound of formula (I) as defined in claim 1 wherein Y is oxygen, sulphur or —N(R^{b})—.

22. A process for preparing a compound of formula (I) as defined in claim 1 wherein Y is oxygen, NR^{b}, sulphur or methylene, comprising reacting a compound of formula (VII) 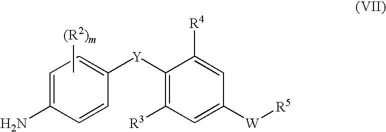
or the corresponding hydrazine of formula (VIIa) formed by reacting a compound of formula (VII) with sodium nitrate which is followed by reduction with a suitable reducing agent 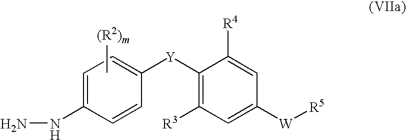
wherein R², R³, R⁴, R⁵ W and m are as defined in claim 1 with a compound of formula (VIII) 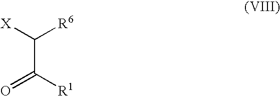
wherein R¹ and R⁶ are as defined in claim 1 and X is hydrogen or a halogen, in the presence of a suitable acid or Lewis acid and followed optionally by interconversion to another compound of formula (I) as defined in claim 1 wherein Y is oxygen, NR^{b}, sulphur or methylene.

23. A process for preparing a compound of formula (I) as defined in claim 1 wherein Y is oxygen, NR^{b} or sulphur, comprising reacting a compound of formula (IX) 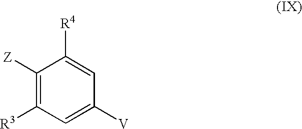
wherein R³ and R⁴ are as defined in claim 1 and where substituent V represents nitro, aldehyde, cyano, carboxyl or derivatives of carboxyls and Z is a suitable leaving group, with a compound of formula (X) 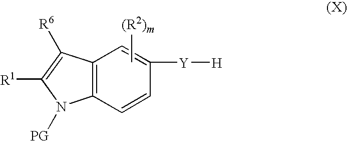
wherein R¹, R⁶, R² and m are as defined in claim 1, PG is hydrogen or a suitable protecting group, and Y is oxygen, sulphur or —N(R^{b})—, optionally in the presence of a suitable base, and optionally, in the presence of copper powder, followed optionally by removal of the protecting group, if present, and optionally by interconversion to another compound of formula (I) as defined in claim 1 wherein Y is oxygen, NR^{b}, or sulphur.
